(19) United States
(12) Patent Application Publication
(10) Pub. No.: US 2011/0212078 A1
(43) Pub. Date: Sep. 1, 2011

(54) SYNTHESIS AND USE OF HETEROCYCLIC ANTIBACTERIAL AGENTS

(76) Inventors: Panduranga Adulla P. Reddy, Walpole, MA (US); Umar Faruk Mansoor, Framingham, MA (US); M. Arshad Siddiqui, Newton, MA (US)

(21) Appl. No.: 13/000,428

(22) PCT Filed: Jun. 24, 2009
(86) PCT No.: PCT/US09/48368

§ 371 Date: May 17, 2011

Related U.S. Application Data
(60) Provisional application No. 61/075,383, filed on Jun. 25, 2008.

(51) Int. Cl. A61K 39/395; C07D 487/04; A61K 31/4985; C07D 209/48; A61K 31/4035; C07D 231/56; A61K 31/416; C07D 403/10; A61K 31/496; C07D 401/10; A61K 31/454; A61P 31/00

(52) U.S. Cl. 424/130.1; 544/350; 514/249; 548/477; 514/417; 548/361.5; 514/405; 544/373; 514/254.09; 546/201; 514/323

(57) ABSTRACT

This invention relates to compounds of the following Formula (I); or a pharmaceutically acceptable salt, solvate, ester or isomer thereof, which is useful for the treatment of diseases or conditions mediated by LpxC.

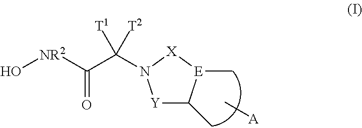

### FIELD OF THE INVENTION

This invention relates generally to heterocycles that can inhibit UDP-3-O—(R-3-hydroxymyristoyl)-N-acetylglucosamine deacetylase (LpxC), and as a result have antimicrobial activity.

### BACKGROUND OF THE INVENTION

Lipid A is the hydrophobic anchor of lipopolysaccharide (LPS) and forms the major lipid component of the outer monolayer of the outer membrane of gram-negative bacteria. Lipid A is required for bacterial growth and inhibition of its biosynthesis is lethal to the bacteria. Furthermore, blocking Lipid A biosynthesis increases the sensitivity of bacteria to other antibiotics.

One of the key enzymes of bacterial lipid A biosynthesis is LpxC. LpxC catalyzes the removal of the N-acetyl group of UDP-3-O—(R-3-hydroxymyristoyl)-N-acetylglucosamine. The LpxC enzyme is essential in gram negative bacteria for the biosynthesis of Lipid A, and it is notably absent from mammalian genomes. Since LpxC is essential for Lipid A biosynthesis and inhibition of Lipid A biosynthesis is lethal to bacteria, inhibitors of LpxC have utility as antibiotics. In addition, the absence of LpxC from mammalian genomes reduces potential toxicity of LpxC inhibitors in mammals. Accordingly, LpxC is an attractive target for antibacterial drug discovery.

U.S. Pat. No. 5,925,659 teaches that certain heterocyclic hydroxamate compounds, in particular oxazoline compounds, have the ability to inhibit LpxC.

WO2004/00744 refers to N-Hydroxyamide derivatives having LpxC inhibitory activity and thus possessing antibacterial activity.

WO2004/062601 also refers to small molecule inhibitors of LpxC.

WO2007/064732 refers to N-Hydroxyamide derivatives having LpxC inhibitory activity and thus possessing antibacterial activity.

WO2008/027466 also refers to small molecule inhibitors of LpxC.

There is a need in the art for small molecule inhibitors of LpxC as potential antibacterial agents.

### SUMMARY OF THE INVENTION

In one aspect, the present invention provides compounds of Formula (I)

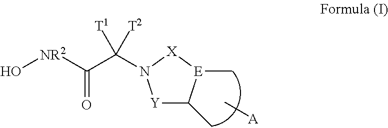

and a pharmaceutically acceptable salts, solvates, esters and prodrugs thereof, where T¹, T², R², X, Y, E, and A are selected independently of each other, wherein:
T¹ and T² are each independently selected from the group consisting of H, alkyl, alkenyl, alkynyl, aryl, cycloalkenyl, cycloalkyl, aralkyl, aralkenyl, aralkenyl, cycloalkenylalkyl, cycloalkenylalkenyl, cyclylalkyl, cyclylalkenyl, heteroaryl, heterocyclenyl, heterocyclyl, heteroaralkyl, heteroaralkenyl, heterocyclenylalkyl, heterocyclenylalkenyl, heterocyclylalkyl, and heterocyclylalkenyl, wherein each of said alkyl, alkenyl, alkynyl, aryl, cycloalkenyl, cycloalkyl, aralkyl, aralkenyl, aralkenyl, cycloalkenylalkyl, cycloalkenylalkenyl, cyclylalkyl, cyclylalkenyl, heteroaryl, heterocyclenyl, heterocyclyl, heteroaralkyl, heteroaralkenyl, heterocyclenylalkyl, heterocyclenylalkenyl, heterocyclylalkyl, and heterocyclylalkenyl can be unsubstituted or substituted with one or more moieties independently selected from the group consisting of H, alkyl, alkenyl, alkynyl, aryl, cycloalkenyl, cycloalkyl, aralkyl, aralkenyl, cycloalkenylalkyl, cycloalkenylalkenyl, cycloalkylalkyl, cycloalkylalkenyl, —OH, O-aryl, O-cycloalkenyl, —O-cycloalkyl, —O-heteroaryl, —O-heterocyclenyl, —O-heterocyclyl, —O-alkyl, —O-alkenyl, —O-alkynyl, —O-aralkyl, —O-aralkenyl, —O-cycloalkenylalkyl, —O-cycloalkylalkyl, —SH, S-aryl, S-cycloalkenyl, —S-cycloalkyl, —S-heteroaryl, —S-heterocyclenyl, —S-heterocyclyl, —S-alkyl, —S-alkenyl, —S-alkynyl, —S-aralkyl, —S-aralkenyl, —S-cycloalkenylalkyl, —S-cycloalkylalkyl, —NH₂, NH-aryl, NH-cycloalkenyl, —NH-cycloalkyl, —NH-heteroaryl, —NH-heterocyclenyl, —NH-heterocyclyl, —NH-alkyl, —NH-alkenyl, —NH-alkynyl, —NH-aralkyl, —NH-aralkenyl, —NH-cycloalkenylalkyl, —NH-cycloalkylalky, further wherein each of said alkyl, alkenyl, alkynyl, aryl, cycloalkeny, cycloalkyl, aralkyl, aralkenyl, aralkenyl, cycloalkenylalkyl, cyclylalkyl, heteroaryl, heterocyclenyl, heterocyclyl, heteroaralkyl, heteroaralkenyl, heterocyclenylalkyl, and heterocyclylalkyl can be unsubstituted or independently substituted from the group consisting of H, alkyl, alkenyl, alkynyl, aryl, cycloalkenyl, cycloalkyl, aralkyl, aralkenyl, cycloalkenylalkyl, and cycloalkylalkyl;
   or

T¹ and T² together with the carbon to which they are attached form a ring selected from the group consisting of spirocyclyl, spirocycloalkenyl, spiroheterocyclyl, and spiroheterocyclenyl, wherein each of said spirocyclyl, spirocycloalkenyl, spiroheterocyclyl, and spiroheterocyclenyl can be unsubstituted or substituted with up to three moieties, independently selected from the group consisting of H, alkyl, alkenyl, alkynyl, aryl, cycloalkenyl, cycloalkyl, aralkyl, aralkenyl, cycloalkenylalkyl, cycloalkylalkyl, —OH, O-aryl, O-cycloalkenyl, —O-cycloalkyl, —O-heteroaryl, —O-heterocyclenyl, —O-heterocyclyl, —O-alkyl, —O-alkenyl, —O-alkynyl, —O-aralkyl, —O-aralkenyl, —O-cycloalkenylalkyl, —O-cycloalkylalkyl, —SH, S-aryl, S-cycloalkenyl, —S-cycloalkyl, —S-heteroaryl, —S-heterocyclenyl, —S-heterocyclyl, —S-alkyl, —S-alkenyl, —S-alkynyl, —S-aralkyl, —S-aralkenyl, —S-cycloalkenylalkyl, —S-cycloalkylalkyl, —NH₂, —NH-aryl, —NH-cycloalkenyl, —NH-cycloalkyl, —NH-heteroaryl, —NH-heterocyclenyl, —NH-heterocyclyl, —NH-alkyl, —NH-alkenyl, —NH-alkynyl, —NH-aralkyl, —NH-aralkenyl, —NH-cycloalkenylalkyl, and —NH-cycloalkylalkyl;

X and Y are independently selected from the group consisting of —CR⁵R⁶, C(O), —S(O)₂, —C(NH)— and NR⁵, wherein each of R⁵ and R⁶ is independently selected from the group consisting of H, alkyl, alkenyl, alkynyl, aryl, cycloalkenyl, cycloalkyl, heteroaryl, heterocyclenyl, heterocyclyl, aralkyl, aralkenyl, cycloalkenylalkyl, cycloalkylalkyl, heteroaralkyl, heterocyclenylalkyl and heterocyclylalkyl, where in each of said alkyl, alkenyl, alkynyl, aryl, cycloalkenyl, cycloalkyl, heteroaryl, heterocyclenyl, heterocyclyl, aralkyl, aralkenyl, cycloalkenylalkyl, cycloalkylalkyl, heteroaralkyl, heterocyclenylalkyl and heterocyclylalkyl can be unsubstituted or substituted with up to three moieties independently selected from the group consisting of H, alkyl, alkenyl, alkynyl, aryl, cycloalkenyl, cycloalkyl, aralkyl, aralkenyl, cycloalkenylalkyl, and cycloalkylalkyl;

E is C, OH, or N;

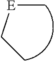

is a six-membered ring selected from the group consisting of aryl, cycloalkenyl, cycloalkyl, heteroaryl, heterocyclenyl and heterocyclyl;

A is selected from the group consisting of alkyl, alkenyl, alkynyl, cycloalkyl, cycloalkenyl, spirocyclyl, spirocycloalkenyl, spiroheterocyclyl, spiroheterocyclenyl, aryl, aralkyl, aralkenyl, cycloalkenylalkyl, cycloalkylalkyl, heteroaryl, heterocyclenyl, heterocyclyl, heteroaralkyl, heterocyclenylalkyl, heterocyclylalkyl, halo, —C(O)R, —C(S)R and —C(NH)R, wherein each of said alkyl, alkenyl, alkynyl, cycloalkyl, cycloalkenyl, spirocyclyl, spirocycloalkenyl, spiroheterocyclyl, spiroheterocyclenyl, aryl, aralkyl, aralkenyl, cycloalkenylalkyl, cycloalkylalkyl, heteroaryl, heterocyclenyl, heterocyclyl, heteroaralkyl, heterocyclenylalkyl, heterocyclylalkyl, is unsubstituted or substituted with up to three moieties independently selected from the group consisting of halo, —OH, O-aryl, O-cycloalkenyl, —O-cycloalkyl, —O-heteroaryl, —O-heterocyclenyl, —O-heterocyclyl, —O-alkyl, —O-alkenyl, —O-alkynyl, —O-aralkyl, —O-aralkenyl, —O-cycloalkenylalkyl, —O-cycloalkylalkyl, —SH, S-aryl, S-cycloalkenyl, —S-cycloalkyl, —S-heteroaryl, —S-heterocyclenyl, —S-heterocyclyl, —S-alkyl, —S-alkenyl, —S-alkynyl, —S-aralkyl, —S-aralkenyl, —S-cycloalkenylalkyl, —S-cycloalkylalkyl, —NH₂, —NH-aryl, —NH-cycloalkenyl, —NH-cycloalkyl, —NH-heteroaryl, —NH-heterocyclenyl, —NH-heterocyclyl, —NH-alkyl, —NH-alkenyl, —NH-alkynyl, —NH-aralkyl, —NH-aralkenyl, —NH-cycloalkenylalkyl, —NH-cycloalkylalkyl, aryl, cycloalkenyl, cycloalkyl, heteroaryl, heterocyclenyl, heterocyclyl, alkyl, alkenyl and alkynyl, wherein each of said aryl, cycloalkenyl, cycloalkyl, heteroaryl, heterocyclenyl, heterocyclyl, alkyl, alkenyl, alkynyl, O-aryl, O-cycloalkenyl, —O-cycloalkyl, —O-heteroaryl, —O-heterocyclenyl, —O-heterocyclyl, —O-alkyl, —O-alkenyl, —O-alkynyl, —O-aralkyl, —O-aralkenyl, —O-cycloalkenylalkyl, —O-cycloalkylalkyl, S-aryl, S-cycloalkenyl, —S-cycloalkyl, —S-heteroaryl, —S-heterocyclenyl, S-heterocyclyl, —S-alkyl, —S-alkenyl, —S-alkynyl, —S-aralkyl, —S-aralkenyl, —S-cycloalkenylalkyl, —S-cycloalkylalkyl, —NH-aryl, —NH-cycloalkenyl, —NH-cycloalkyl, —NH-heteroaryl, —NH-heterocyclenyl, —NH-heterocyclyl, —NH-alkenyl, —NH-alkynyl, —NH-aralkyl, —NH-aralkenyl, —NH-cycloalkenylalkyl, —NH-cycloalkylalkyl, aryl, cycloalkenyl, cycloalkyl, heteroaryl, heterocyclenyl, heterocyclyl, alkyl, alkenyl, alkynyl can be unsubstituted or substituted with up to three moieties independently selected from the group consisting of halo, —OH, O-aryl, O-cycloalkenyl, —O-cycloalkyl, —O-heteroaryl, —O-heterocyclenyl, —O-heterocyclyl, —O-alkyl, —O-alkenyl, —O-alkynyl, —O-aralkyl, —O-aralkenyl, —O-cycloalkenylalkyl, —O-cycloalkylalkyl, —SH, S-aryl, S-cycloalkenyl, —S-cycloalkyl, —S-heteroaryl, —S-heterocyclenyl, —S-heterocyclyl, —S-alkyl, S-alkenyl, —S-alkynyl, —S-aralkyl, —S-aralkenyl, —S-cycloalkenylalkyl, and —S-cycloalkylalkyl, —NH₂, —NH-aryl, —NH-cycloalkenyl, —NH-cycloalkyl, —NH-heteroaryl, —NH-heterocyclenyl, —NH-heterocyclyl, —NH-alkyl, —NH-alkenyl, —NH-alkynyl, —NH-aralkyl, —NH-aralkenyl, —NH-cyclenylalkyl, —NH-cycloalkylalkyl, aryl, cycloalkenyl, cycloalkyl, heteroaryl, heterocyclenyl, heterocyclyl, alkyl, alkenyl, alkynyl, wherein each of said O-aryl, O-cycloalkenyl, —O-cycloalkyl, —O-heteroaryl, —O-heterocyclenyl, —O-heterocyclyl, O-alkyl, —O-alkenyl, —O-alkynyl, —O-aralkyl, —O-aralkenyl, —O-cycloalkenylalkyl, —O-cycloalkylalkyl, S-aryl, S-cycloalkenyl, —S-cycloalkyl, —S-heteroaryl, —S-heterocyclenyl, —S-heterocyclyl, —S-alkyl, —S-alkenyl, —S-alkynyl, —S-aralkyl, —S-aralkenyl, —S-cycloalkenylalkyl, —S-cycloalkylalkyl, —NH-aryl, —NH-cycloalkenyl, —NH-cycloalkyl, —NH-heteroaryl, —NH-heterocyclenyl, —NH-heterocyclyl, —NH-alkyl, —NH-alkenyl, —NH-alkynyl, —NH-aralkyl, —NH-aralkenyl, —NH-cycloalkenylalkyl, —NH-cycloalkylalkyl, aryl, cycloalkenyl, cycloalkyl, heteroaryl, heterocyclenyl, heterocyclyl, alkyl, alkenyl and alkynyl can be unsubstituted or substituted with one or more moieties independently selected from the group consisting of halo, —OH, O-a O-cycloalkenyl, —O-cycloalkyl, —O-heteroaryl, —O-heterocyclenyl, —O-heterocyclyl, —O-alkyl, —O-alkenyl, —O-alkynyl, —O-aralkyl, —O-aralkenyl, —O-cycloalkenylalkyl, —O-cycloalkylalkyl, —SH, S-aryl, S-cycloalkenyl, —S-cycloalkyl, —S-heteroaryl, —S-heterocyclenyl, —S-heterocyclyl, —S-alkyl, —S-alkenyl, —S-alkynyl, —S-aralkyl, —S-aralkenyl, —S-cycloalkenylalkyl, —S-cycloalkylalkyl, —NH-aryl, —NH-cycloalkenyl, —NH-cycloalkyl, —NH-heteroaryl, —NH-heterocyclenyl, —NH-—NH-alkyl, —NH-alkenyl, —NH-alkynyl, —NH-aralkyl, —NH-aralkenyl, —NH-cycloalkenylalkyl, —NH-cycloalkylalkyl, aryl, cycloalkenyl, cycloalkyl, heteroaryl, heterocyclenyl, heterocyclyl, alkyl, alkenyl and alkynyl;

R² is independently selected from the group consisting of H, alkyl, alkenyl, alkynyl, cycloalkyl, cycloalkenyl, aryl, heteroaryl, heterocyclyl and heterocyclenyl, wherein each of said alkyl, alkenyl, alkynyl, cycloalkyl, cycloakenyl, aryl, heteroaryl, heterocyclyl, and heterocyclenyl can be unsubstituted or substituted with up to three moieties independently selected from the group consisting of halogen, alkyl, alkenyl, alkynyl, aryl, cycloalkenyl, cycloalkyl, aralkyl, aralkenyl, cycloalkenylalkyl, cycloalkylalkyl, —OH, O-aryl, O-cycloalkenyl, —O-cycloalkyl, —O-heteroaryl, —O-heterocyclenyl, —O-heterocyclyl, —O-alkyl, —O-alkenyl, —O-alkynyl, —O-aralkyl, —O-aralkenyl, —O-cycloalkenylalkyl, —O-cycloalkylalkyl, —SH, S-aryl, S-cycloalkenyl, —S-cycloalkyl, —S-heteroaryl, —S-heterocyclenyl, —S-heterocyclyl, —S-alkyl, —S-alkenyl, —S-alkynyl, —S-aralkyl, —S-aralkenyl, —S-cycloalkenylalkyl, —S-cycloalkylalkyl, —NH₂, —NH-aryl, —NH-cycloalkenyl, —NH-cycloalkyl, —NH-heteroaryl, —NH-heterocyclenyl, —NH-heterocyclyl, —NH-alkyl, —NH-alkenyl, —NH-alkynyl, —NH-aralkyl, —NH-aralkenyl, —NH-cycloalkenylalkyl, and —NH-cycloalkylalkyl.

The compound according to claim 1, wherein T¹ and T² are each independently selected from the group consisting of H and (C₁-C₈)alkyl, wherein said (C₁-C₈)alkyl, can be unsubstituted or substituted with one or more moieties independently selected from the group consisting of alkyl, —OH, NH₂,

In another aspect, the invention provides a novel class of compounds as inhibitors of LpxC, methods of preparing such compounds, pharmaceutical compositions comprising one or more such compounds, methods of preparing pharmaceutical formulations comprising one or more such compounds, and methods of treatment, prevention, inhibition or amelioration of one or more diseases associated with LpxC, using such compounds or pharmaceutical compositions.

In another aspect, the invention provides a method of treating microbial infections.

In a further aspect, the invention provides compositions comprising one or more compounds of Formula (I), an additional therapeutic agent for treating LpxC receptor mediated disease, and a pharmaceutical carrier.

### DETAILED DESCRIPTION OF THE INVENTION

In its several embodiments, the present invention provides a novel class of inhibitors of LpxC, pharmaceutical compositions containing one or more of the compounds, methods of preparing pharmaceutical formulations comprising one or more such compounds, and methods of treatment, prevention or amelioration of microbial infections.

In one embodiment, the present invention provides compounds, which are represented by structural Formula (I):

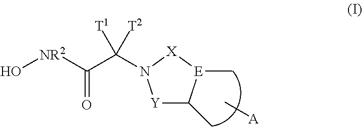

In one embodiment,

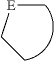

is phenyl, wherein E is C or CH.

In another embodiment,

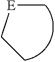

is piperazine, wherein E is N.

In one embodiment, A is alkynyl, wherein said alkynyl is substituted with phenyl, wherein said phenyl can be unsubstituted or substituted with an additional phenyl.

In another embodiment, A is ethynyl, wherein said ethynyl is substituted with phenyl, wherein said phenyl can be unsubstituted or substituted with an additional phenyl.

In still another embodiment, A is ethynyl, wherein said ethynyl is substituted with phenyl, wherein said phenyl can be unsubstituted or para substituted with an additional phenyl.

In yet another embodiment, A is ethynyl, wherein said ethynyl is substituted with phenyl, wherein said phenyl can be unsubstituted or meta substituted with an additional phenyl.

In one embodiment, A is phenyl, wherein said phenyl can be unsubstituted car substituted with alkynyl, wherein said alkynyl is substituted with phenyl.

In another embodiment, A is phenyl, wherein said phenyl can be unsubstituted or substituted with ethynyl, wherein said ethynyl is substituted with an additional phenyl.

In yet another embodiment, A is phenyl, wherein said phenyl can be unsubstituted or substituted with an additional phenyl.

In still another embodiment, A is phenyl, wherein said phenyl can be unsubstituted or para substituted with an additional phenyl.

In one embodiment, A is aralkyl, wherein said aralkyl is substituted with alkynyl, wherein said alkynyl is substituted with aryl.

In another embodiment, A is phenylalkyl, wherein said phenylalkyl is substituted with alkynyl, wherein said alkynyl is substituted with aryl,

In yet another embodiment, A is phenylmethyl, wherein said phenylmethyl is substituted with alkynyl, wherein said alkynyl is substituted with aryl.

In still another embodiment, A is phenylmethyl, wherein said phenylmethyl is substituted with ethynyl, wherein said ethynyl is substituted with aryl.

In another embodiment, A is phenylmethyl, wherein said phenylmethyl is substituted with ethynyl, wherein said ethynyl is substituted with phenyl.

In still another embodiment, A is phenylmethyl, wherein said phenylmethyl is para substituted with ethynyl, wherein said ethynyl is substituted with phenyl.

In yet another embodiment, A is phenylmethyl, wherein said phenylmethyl is para substituted with phenyl.

In one embodiment, A is piperidinyl, wherein said piperidinyl is substituted with phenyl, wherein said phenyl is substituted with an additional phenyl.

In another embodiment, A is piperidinyl, wherein said piperidinyl is para substituted with phenyl, wherein said phenyl is substituted with an additional phenyl.

In still another embodiment, A is piperidinyl, wherein said piperidinyl is para substituted with phenyl, wherein said phenyl is para substituted with an additional phenyl.

In yet another embodiment, A is piperidinyl, wherein said piperidinyl is substituted with phenyl, wherein said phenyl is substituted with alkynyl, wherein said alkynyl is substituted with an additional phenyl.

In another embodiment, A is piperidinyl, wherein said piperidinyl is para substituted with phenyl, wherein said phenyl is substituted with alkynyl, wherein said alkynyl is substituted with phenyl.

In still another embodiment, A is piperidinyl, wherein said piperidinyl is para substituted with phenyl, wherein said phenyl is para substituted with alkynyl, wherein said alkynyl is substituted with an additional phenyl.

In yet another embodiment, A is piperidinyl, wherein said piperidinyl is para substituted with phenyl, wherein said phenyl is para substituted with ethynyl, wherein said ethynyl is substituted with an additional phenyl.

In one embodiment, A is piperazinyl, wherein said piperazinyl is substituted with phenyl.

In another embodiment, A is piperazinyl, wherein said piperazinyl is para substituted with phenyl.

In still another embodiment, A is piperazinyl, wherein said piperazinyl is substituted with phenyl, further wherein said phenyl is substituted with alkynyl, wherein said alkynyl is substituted with phenyl.

In yet another embodiment, A is piperazinyl, wherein said piperazinyl is substituted with phenyl, wherein said phenyl is substituted with alkynyl, wherein said alkynyl is substituted with an additional phenyl.

In another embodiment, A is piperazinyl, wherein said piperazinyl is substituted with phenyl, wherein said phenyl is substituted with ethynyl, wherein said ethynyl is substituted with an additional phenyl.

In still another embodiment, A is piperazinyl, wherein said piperazinyl is para substituted with phenyl, wherein said phenyl is substituted with ethynyl, wherein said ethynyl is substituted with an additional phenyl.

In yet another embodiment, A is piperazinyl, wherein said piperazinyl is substituted with phenyl, wherein said phenyl is substituted with an additional phenyl.

In another embodiment, A is piperazinyl, wherein said piperazinyl is para substituted with phenyl, wherein said phenyl is substituted with an additional phenyl.

In still another embodiment, A is piperazinyl, wherein said piperazinyl is pare substituted with phenyl, wherein said phenyl is para substituted with an additional phenyl.

In still another embodiment, A is piperazinyl, wherein said piperazinyl is para substituted with phenyl, wherein said phenyl is para substituted with aralkylcarbonyl.

In yet another embodiment, A is piperazinyl, wherein said piperazinyl is para substituted with phenyl, further wherein said phenyl is para substituted with phenylmethylcarbonyl.

In one embodiment, A is halo.

In another embodiment, A is bromo.

In yet another embodiment, A is chloro.

In one embodiment, the Compounds of Formula (I) have the Formulae (IA, IB, IC, ID, IE, IIA, IIB, IIC, IID, and HE):

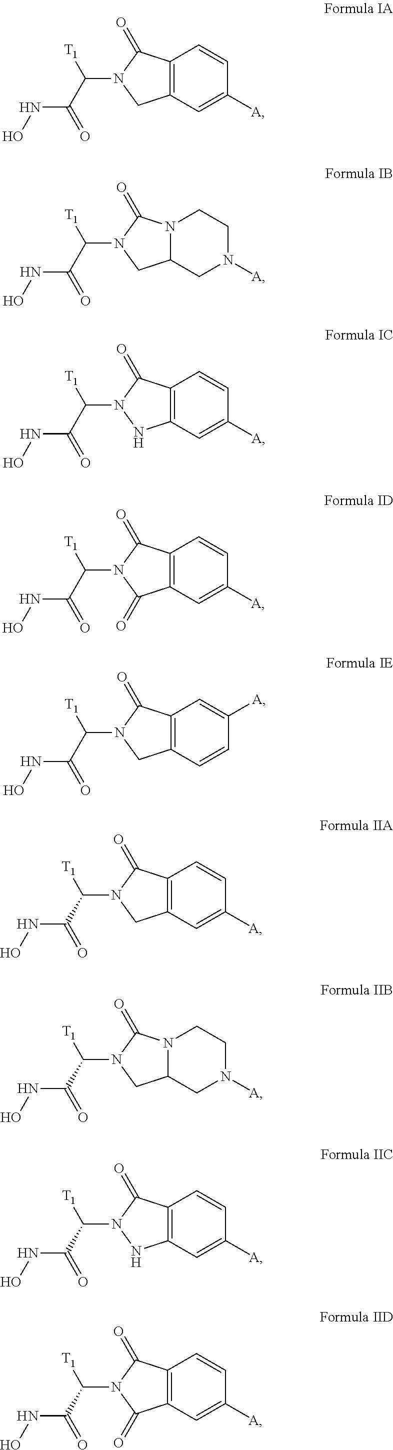

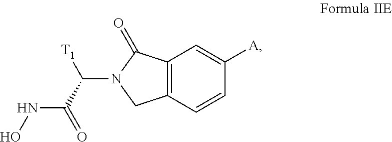

wherein T¹ and A are as defined above for the Compounds of Formula (I).

In one embodiment, T¹ is

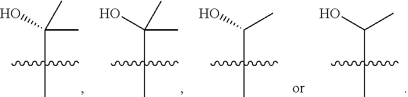

In another embodiment; T¹ is

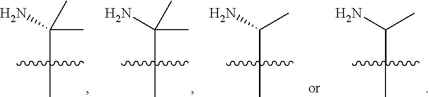

In one embodiment, A is

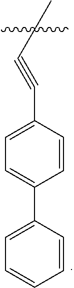

In another embodiment, A is

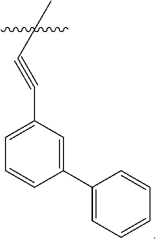

In one embodiment, A is

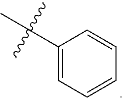

In another embodiment, A is

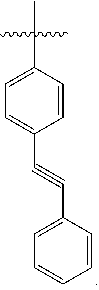

In still another embodiment, A is

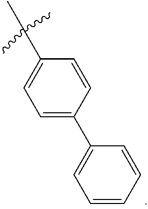

In one embodiment, A is

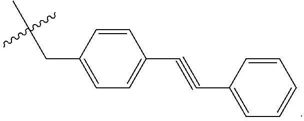

In another embodiment, A is

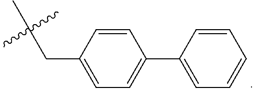

In one embodiment. A is

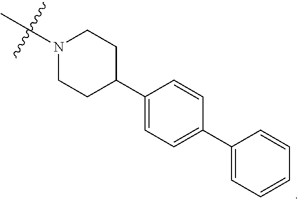

In one embodiment, A is

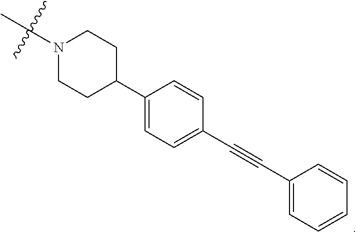

in another embodiment, A is

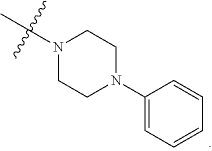

In still another embodiment, A is

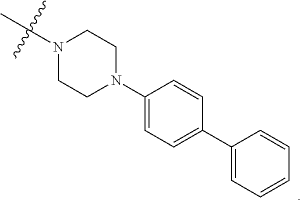

In yet another embodiment, A is

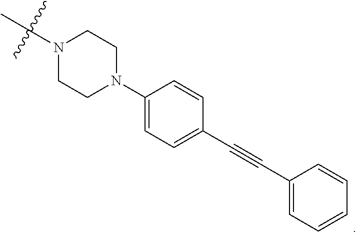

In another embodiment, A is

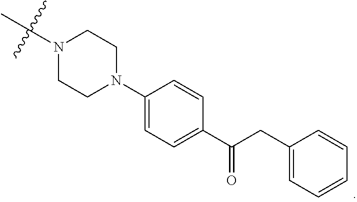

In yet another embodiment, A is Br or Cl.

In one embodiment, the compound of Formula (I) is:

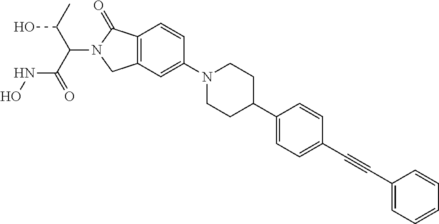

or a pharmaceutically acceptable salt, solvate or ester thereof.

In another embodiment, the compound of Formula (I) is:

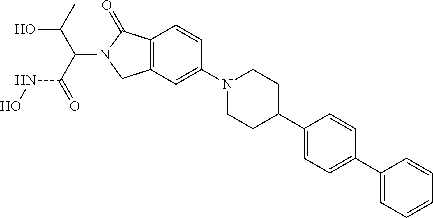

or a pharmaceutically acceptable salt, solvate or ester thereof.

In one embodiment, the compound of Formula (I) is:

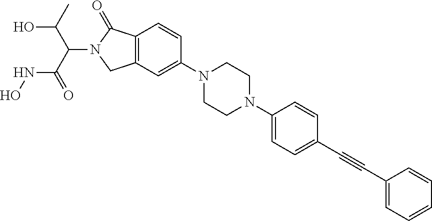

or a pharmaceutically acceptable salt, solvate or ester thereof.

In another embodiment, the compound of Formula (I) is:

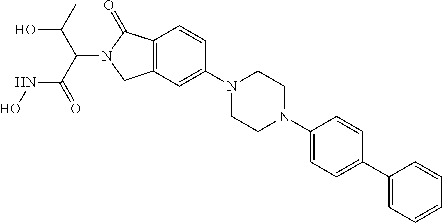

or a pharmaceutically acceptable salt, solvate or ester thereof.

In still another embodiment, the compound of Formula (I) is:

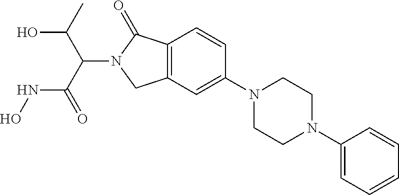

or a pharmaceutically acceptable salt, solvate or ester thereof.

In yet another embodiment, the compound of Formula (I) is:

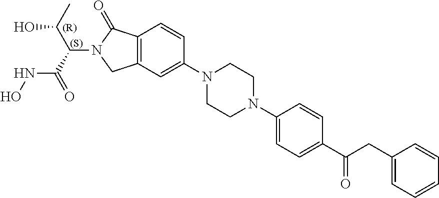

or a pharmaceutically acceptable salt, solvate or ester thereof,

In one embodiment, the compound of Formula (I) is:

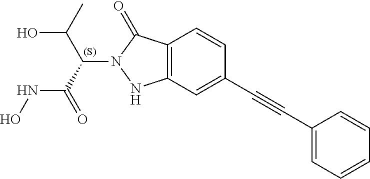

or a pharmaceutically acceptable salt, solvate or ester thereof.

In another embodiment, the compound of Formula (I) is:

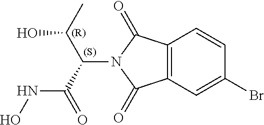

or a pharmaceutically acceptable salt, solvate or ester thereof.

In yet another embodiment, the compound of Formula (I) is:

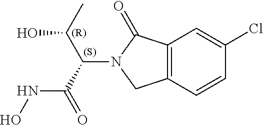

or a pharmaceutically acceptable salt, solvate or ester thereof.

Non-limiting examples of compounds of Formula I include:

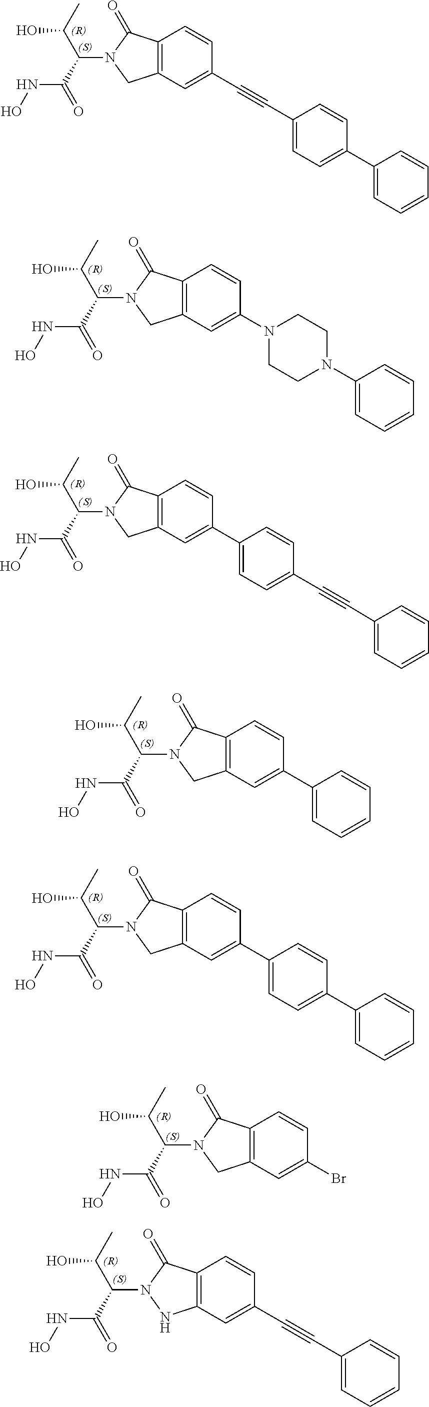

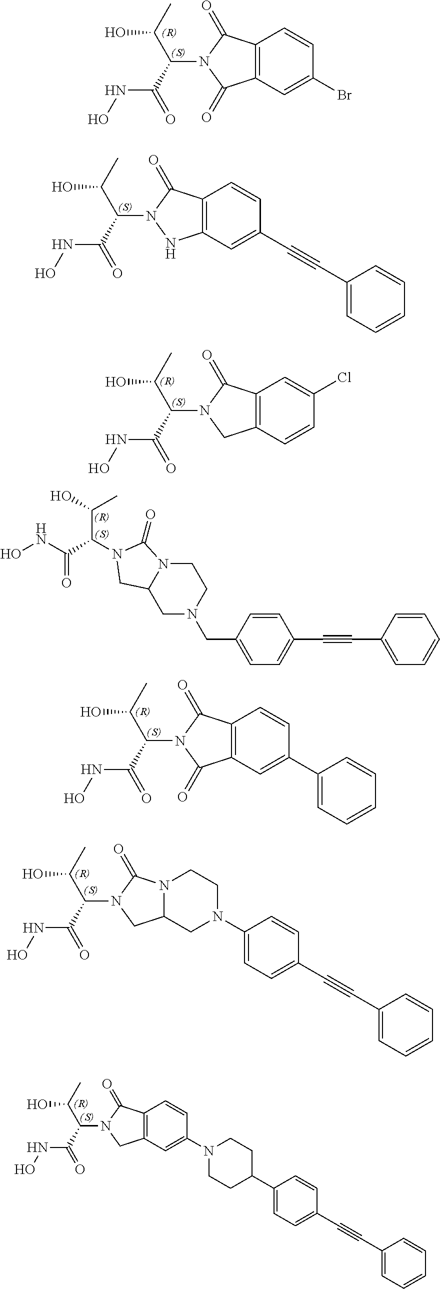

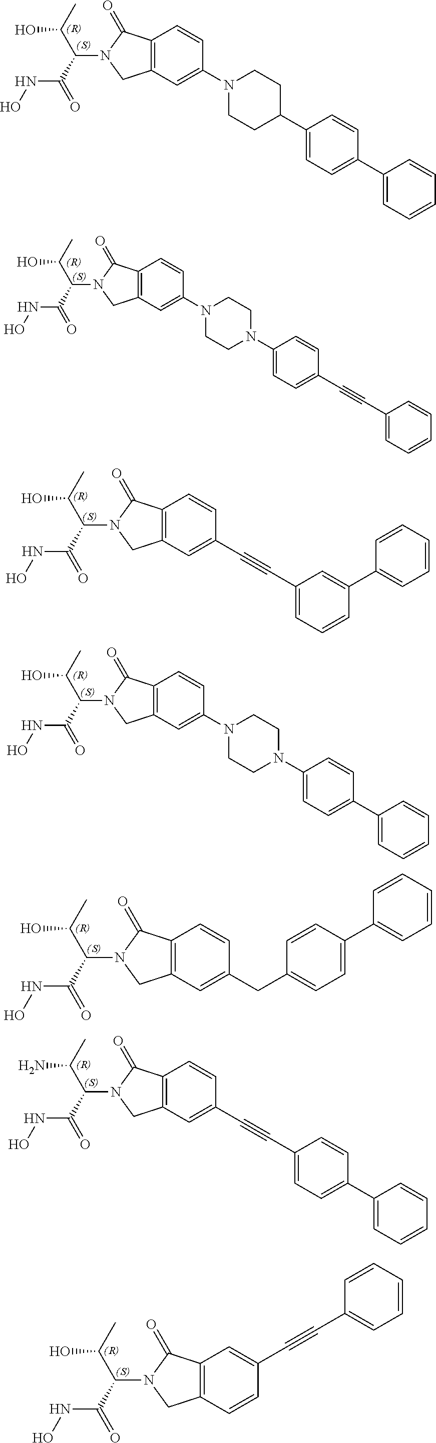

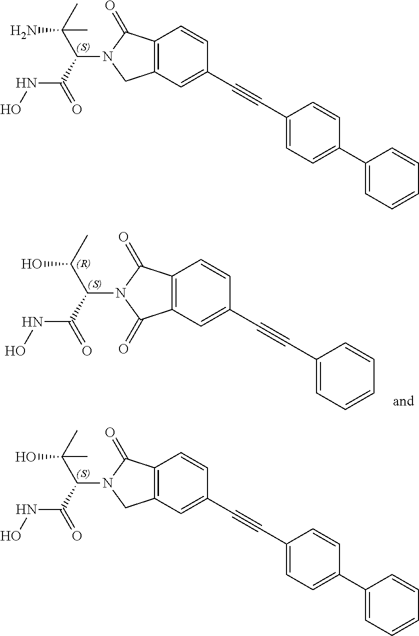

or a pharmaceutically acceptable salt, solvate or ester thereof.

As used above, and throughout this disclosure, the following terms, unless otherwise indicated, shall be understood to have the following meanings;

“Patient//subject” includes both human and animals.

“Mammal” means humans and other mammalian animals.

“Alkyl” means an aliphatic hydrocarbon group which may be straight or branched and comprising about 1 to about 20 carbon atoms in the chain. Preferred alkyl groups contain about 1 to about 12 carbon atoms in the chain. More preferred alkyl groups contain about 1 to about 6 carbon atoms in the chain. Branched means that one or more lower alkyl groups such as methyl, ethyl or propyl, are attached to a linear alkyl chain. “Lower alkyl” means a group having about 1 to about 6 carbon atoms in the chain which may be straight or branched. The term “substituted alkyl” means that the alkyl group may be substituted by one or more substituents which may be the same or different, each substituent being independently selected from the group consisting of halo, alkyl, aryl, cycloalkyl, cyano, hydroxy, alkoxy, alkylthio, amino, —NH(alkyl), —NH(cycloalkyl), —N(alkyl)₂, carboxy and —C(O)O-alkyl. Non-limiting examples of suitable alkyl groups include methyl, ethyl, n-propyl, isopropyl and t-butyl. The term “Fluoroalkyl” means an alkyl group in which alkyl is as previously described wherein one or more hydrogens are replaced with fluorine atoms.

“Alkenyl” means an aliphatic hydrocarbon group containing at least one carbon-carbon double bond and which may be straight or branched and comprising about 2 to about 15 carbon atoms in the chain. Preferred alkenyl groups have about 2 to about 12 carbon atoms in the chain; and more preferably about 2 to about 6 carbon atoms in the chain. Branched means that one or more lower alkyl groups such as methyl, ethyl or propyl, are attached to a linear alkenyl chain. “Lower alkenyl” means about 2 to about 6 carbon atoms in the chain which may be straight or branched. Non-limiting examples of suitable alkenyl groups include ethenyl, propenyl, n-butenyl, 3-methylbut-2-enyl, n-pentenyl, octenyl and decenyl.

“Alkynyl” means an aliphatic hydrocarbon group containing at least one carbon-carbon triple bond and which may be straight or branched and comprising about 2 to about 15 carbon atoms in the chain. Preferred alkynyl groups have about 2 to about 12 carbon atoms in the chain; and more preferably about 2 to about 4 carbon atoms in the chain. Branched means that one or more lower alkyl groups such as methyl, ethyl or propyl, are attached to a linear alkynyl chain. “Lower alkynyl” means about 2 to about 6 carbon atoms in the chain which may be straight or branched. Non-limiting examples of suitable alkynyl groups include ethynyl, propynyl, 2-butynyl and 3-methylbutynyl. The term “substituted alkynyl” means that the alkynyl group may be substituted by one or more substituents which may be the same or different, each substituent being independently selected from the group consisting of alkyl, aryl and cycloalkyl.

“Aryl” means an aromatic monocyclic or multicyclic ring system comprising about 6 to about 14 carbon atoms, preferably about 6 to about 10 carbon atoms. The aryl group can be optionally substituted with one or more “ring system substituents” which may be the same or different, and are as defined herein, Non-limiting examples of suitable aryl groups include phenyl and naphthyl.

“Heteroaryl” means an aromatic monocyclic or multicyclic ring system comprising about 5 to about 14 ring atoms, preferably about 5 to about 10 ring atoms, in which one or more of the ring atoms is an element other than carbon, for example nitrogen, oxygen or sulfur, alone or in combination. Preferred heteroaryls contain about 5 to about 6 ring atoms. The “heteroaryl” can be optionally substituted by one or more “ring system substituents” which may be the same or different, and are as defined herein. The prefix aza, oxa or thia before the heteroaryl root name means that at least a nitrogen, oxygen or sulfur atom respectively, is present as a ring atom. A nitrogen atom of a heteroaryl can be optionally oxidized to the corresponding N-oxide. Non-limiting examples of suitable heteroaryls include pyridyl, pyrazinyl, furanyl, thienyl, pyrimidinyl, pyridone (including N-substituted pyridones), isoxazolyl, isothiazolyl, oxazolyl, thiazolyl, pyrazolyl, furazanyl, pyrrolyl, pyrazolyl, triazolyl, 1,2,4-thiadiazolyl, pyrazinyl, pyridazinyl, quinoxalinyl, phthalazinyl, oxindolyl, imidazo[1,2-a]pyridinyl, imidazo[2,1-b]thiazolyl, benzofurazanyl, indolyl, azaindolyl, benzimidazolyl, benzothienyl, quinolinyl, imidazolyl, thienopyridyl, quinazolinyl, thienopyrimidyl, pyrrolopyridyl, imidazopyridyl, isoquinolinyl, benzoazaindolyl, 1,2,4-triazinyl, benzothiazolyl and the like. The term “heteroaryl” also refers to partially saturated heteroaryl moieties such as, for example, tetrahydroisoquinolyl, tetrahydroquinolyl and the like.

“Aralkyl” or “arylalkyl” means an aryl-alkyl-group in which the aryl and alkyl are as previously described. Preferred aralkyls comprise a lower alkyl group. Non-limiting examples of suitable aralkyl groups include benzyl, 2-phenethyl and naphthalenylmethyl. The bond to the parent moiety is through the alkyl.

“Alkylaryl” means an alkyl-aryl-group in which the alkyl and aryl are as previously described. Preferred alkylaryls comprise a lower alkyl group. Non-limiting example of a suitable alkylaryl group is tolyl. The bond to the parent moiety is through the aryl.

“Cycloalkyl” means a non-aromatic mono- or multicyclic ring system comprising about 3 to about 10 carbon atoms, preferably about 5 to about 10 carbon atoms. Preferred cycloalkyl rings contain about 5 to about 7 ring atoms. The cycloalkyl can be optionally substituted with one or more “ring system substituents” which may be the same or different, and are as defined above. Non-limiting examples of suitable monocyclic cycloalkyls include cyclopropyl, cyclopentyl, cyclohexyl, cycloheptyl and the like. Non-limiting examples of suitable multicyclic cycloalkyls include 1-decalinyl, norbornyl, adamantyl and the like, as well as partially saturated species such as, for example, indanyl, tetrahydronaphthyl and the like.

“Cycloalkenyl” means a non-aromatic mono or multicyclic ring system comprising about 3 to about 10 carbon atoms, preferably about 5 to about 10 carbon atoms which contains at least one carbon-carbon double bond. Preferred cycloalkenyl rings contain about 5 to about 7 ring atoms. The cycloalkenyl can be optionally substituted with one or more “ring system substituents” which may be the same or different, and are as defined above. Non-limiting examples of suitable monocyclic cycloalkenyls include cyclopentenyl, cyclohexenyl, cyclohepta-1,3-dienyl, and the like. Non-limiting example of a suitable multicyclic cycloalkenyl is norbornylenyl.

“Haloalkyl” means an alkyl as defined above wherein one or more hydrogen atoms on the alkyl is replaced by a halo group defined above. Non-limiting examples include trifluoromethyl, 2,2,2-trifluoroethyl, 2-chloropropyl and alike.

“Haloalkoxy” means an alkoxy group as defined below wherein one or more hydrogen atoms on the alkoxy is replaced by a halo/halogen group defined above. Non-limiting examples include trifluoromethoxy (CF₃O—), difluoromethoxy (CHF₂O—), 2,2,2-trifluoroethoxy (CF₃CH₂O—), 2-chloropropoxy (CH₃CH(Cl)CH₂O—) and alike.

“Halogen” or “halo” means fluorine, chlorine, bromine, or iodine. Preferred are fluorine, chlorine and bromine.

“Ring system substituent” means a substituent attached to an aromatic or non-aromatic ring system which, for example, replaces an available hydrogen on the ring system. Ring system substituents may be the same or different, each being independently selected from the group consisting of alkyl, alkenyl, alkynyl, aryl, heteroaryl, aralkyl, alkylaryl, heteroaralkyl, heteroarylalkenyl, heteroarylalkynyl, alkylheteroaryl, hydroxy, hydroxyalkyl, alkoxy; aryloxy, aralkoxy, acyl, aroyl, halo, nitro, cyano, carboxy, alkoxycarbonyl, aryloxycarbonyl, aralkoxycarbonyl, alkylsulfonyl, arylsulfonyl, heteroarylsulfonyl, alkylthio, arylthio, heteroarylthio, aralkylthio, heteroaralkylthio, cycloalkyl, heterocyclyl, —C(═N—CN)—NH₂, —C(═NH)—NH₂, —C(═NH)—NH(alkyl), Y₁Y₂N—, Y₁Y₂N-alkyl-, Y₁Y₂NC(O)—, Y₁Y₂NSO₂— and —SO₂NY₁Y₂, wherein Y₁ and Y₂ can be the same or different and are independently selected from the group consisting of hydrogen, alkyl, aryl, cycloalkyl, and aralkyl. “Ring system substituent” may also mean a single moiety which simultaneously replaces two available hydrogens on two adjacent carbon atoms (one H on each carbon) on a ring system. Examples of such moiety are methylene dioxy, ethylenedioxy, —C(CH₃)₂— and the like which form moieties such as, for example:

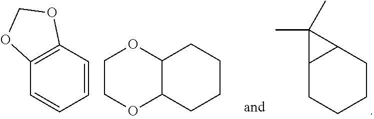

“Heterocyclyl” means a non-aromatic saturated monocyclic or multicyclic ring system comprising about 3 to about 10 ring atoms, preferably about 5 to about 10 ring atoms, in which one or more of the atoms in the ring system is an element other than carbon, for example nitrogen, oxygen or sulfur, alone or in combination. There are no adjacent oxygen and/or sulfur atoms present in the ring system. Preferred heterocyclyls contain about 5 to about 6 ring atoms. The prefix aza, oxa or thia before the heterocyclyl root name means that at least a nitrogen, oxygen or sulfur atom respectively is present as a ring atom. Any —NH in a heterocyclyl ring may exist protected such as, for example, as an —N(Boc), —N(CBz), —N(Tos) group and the like; such protections are also considered part of this invention. The heterocyclyl can be optionally substituted by one or more “ring system substituents” which may be the same or different, and are as defined herein. The nitrogen or sulfur atom of the heterocyclyl can be optionally oxidized to the corresponding N-oxide, S-oxide or S,S-dioxide. Non-limiting examples of suitable monocyclic heterocyclyl rings include piperidyl, pyrrolidinyl, piperazinyl, morpholinyl, thiomorpholinyl, thiazolidinyl, 1,4-dioxanyl, tetrahydrofuranyl, tetrahydrothiophenyl, lactam, lactone, and the like.

“Spiro ring systems” have two or more rings linked by one common atom. Preferred spiro ring systems include spiroheteroaryl, spiroheterocyclenyl, spiroheterocyclyl, spirocycloalkyl, spirocycloalkenyl, and spiroaryl. Non-limiting examples of suitable spiro ring systems include

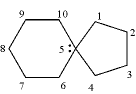

spiro[4.5]decane,

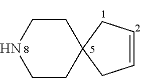

8-azaspiro[4.5]dec-2-ene, and

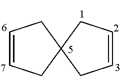

spiro[4.4]nona-2,7-diene.

“Heterocyclenyl” means a partially unsaturated monocyclic or partially unsaturated multicyclic ring system comprising about 5 to about 14 ring atoms, preferably about 5 to about 10 ring atoms, in which one or more of the ring atoms is an element other than carbon, for example nitrogen, oxygen or sulfur, alone or in combination. Preferred heterocyclenyls contain about 5 to about 6 ring atoms and 1-3 double bonds. Preferred heterocyclenyls also contain at least one —C═N as part of the ring. The “heterocyclenyl” can be optionally substituted by one or more “ring system substituents” which may be the same or different, and are as defined herein. The prefix aza, oxa or thia before the heterocyclenyl root name means that at least a nitrogen, oxygen or sulfur atom respectively, is present as a ring atom. The nitrogen or sulfur atom of the heteroaryl can be optionally oxidized to the corresponding N-oxide, S-oxide or S,S-dioxide. Non-limiting examples of suitable heterocyclenyls include dihydroimidazole, dihydrooxazole, dihydrooxadiazole, dihydrothiazole, and the like.

It should be noted that in hetero-atom containing ring systems of this invention, there are no hydroxyl groups on carbon atoms adjacent to a N, O or S, as well as there are no N or S groups on carbon adjacent to another heteroatom. Thus, for example, in the ring:

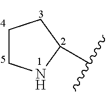

there is no —OH attached directly to carbons marked 2 and 5.

It should also be noted that tautomeric forms such as, for example, the moieties:

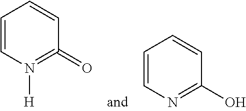

are considered equivalent in certain embodiments of this invention.

“Alkynylalkyl” means an alkynyl-alkyl-group in which the alkynyl and alkyl are as previously described. Preferred alkynylalkyls contain a lower alkynyl and a lower alkyl group. The bond to the parent moiety is through the alkyl. Non-limiting examples of suitable alkynylalkyl groups include propargylmethyl.

“Heteroaralkyl” means a heteroaryl-alkyl-group in which the heteroaryl and alkyl are as previously described. Preferred heteroaralkyls contain a lower alkyl group. Non-limiting examples of suitable aralkyl groups include pyridylmethyl, and quinolin-3-ylmethyl. The bond to the parent moiety is through the alkyl.

“Hydroxyalkyl” means a HO-alkyl-group in which alkyl is as previously defined. Preferred hydroxyalkyls contain lower alkyl. Non-limiting examples of suitable hydroxyalkyl groups include hydroxymethyl and 2-hydroxyethyl.

“Acyl” means an H—C(O)—, alkyl-C(O)— or cycloalkyl-C(O)—, group in which the various groups are as previously described. The bond to the parent moiety is through the carbonyl. Preferred acyls contain a lower alkyl. Non-limiting examples of suitable acyl groups include formyl, acetyl and propanoyl,

“Aroyl” means an aryl-C(O)— group in which the aryl group is as previously described. The bond to the parent moiety is through the carbonyl. Non-limiting examples of suitable groups include benzoyl and 1-naphthoyl.

“Alkoxy” means an alkyl-O— group in which the alkyl group is as previously described. Non-limiting examples of suitable alkoxy groups include methoxy, ethoxy, n-propoxy, isopropoxy and n-butoxy. The bond to the parent moiety is through the ether oxygen.

“Aryloxy” means an aryl-O— group in which the aryl group is as previously described Non-limiting examples of suitable aryloxy groups include phenoxy and naphthoxy. The bond to the parent moiety is through the ether oxygen.

“Aralkyloxy” means an aralkyl-O— group in which the aralkyl group is as previously described. Non-limiting examples of suitable aralkyloxy groups include benzyloxy and 1- or 2-naphthalenemethoxy. The bond to the parent moiety is through the ether oxygen.

“Alkylthio” means an alkyl-S— group in which the alkyl group is as previously described. Non-limiting examples of suitable alkylthio groups include methylthio and ethylthio. The bond to the parent moiety is through the sulfur.

“Arylthio” means an aryl-S— group in which the aryl group is as previously described. Non-limiting examples of suitable arylthio groups include phenylthio and naphthylthio. The bond to the parent moiety is through the sulfur.

“Aralkylthio” means an aralkyl-S— group in which the aralkyl group is as previously described. Non-limiting example of a suitable aralkylthio group is benzylthio. The bond to the parent moiety is through the sulfur.

“Alkoxycarbonyl” means an alkyl-O—CO— group. Non-limiting examples of suitable alkoxycarbonyl groups include methoxycarbonyl and ethoxycarbonyl. The bond to the parent moiety is through the carbonyl.

“Aryloxycarbonyl” means an aryl-O—C(O)— group, Non-limiting examples of suitable aryloxycarbonyl groups include phenoxycarbonyl and naphthoxycarbonyl. The bond to the parent moiety is through the carbonyl.

“Aralkoxycarbonyl” means an aralkyl-O—C(O)— group. Non-limiting example of a suitable aralkoxycarbonyl group is benzyloxycarbonyl. The bond to the parent moiety is through the carbonyl.

“Alkylsulfonyl” means an alkyl-S(O₂)— group. Preferred groups are those in which the alkyl group is lower alkyl. The bond to the parent moiety is through the sulfonyl.

“Arylsulfonyl” means an aryl-S(O₂)— group. The bond to the parent moiety is through the sulfonyl.

The term “substituted” means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds. By “stable compound” or “stable structure” is meant a compound that is sufficiently robust to survive isolation to a useful degree of purity from a reaction mixture, and formulation into an efficacious therapeutic agent.

The term “optionally substituted” means optional substitution with the specified groups, radicals or moieties.

The term “isolated” or “in isolated form” for a compound refers to the physical state of said compound after being isolated from a synthetic process or natural source or combination thereof. The term “purified” or “in purified form” for a compound refers to the physical state of said compound after being obtained from a purification process or processes described herein or well known to the skilled artisan, in sufficient purity to be characterizable by standard analytical techniques described herein or well known to the skilled artisan.

It should also be noted that any carbon as well as heteroatom with unsatisfied valences in the text, schemes, examples and Tables herein is assumed to have the sufficient number of hydrogen atom(s) to satisfy the valences.

When a functional group in a compound is termed “protected”, this means that the group is in modified form to preclude undesired side reactions at the protected site when the compound is subjected to a reaction. Suitable protecting groups will be recognized by those with ordinary skill in the art as well as by reference to standard textbooks such as, for example, T. W. Greene et al, Protective Groups in organic Synthesis (1991), Wiley, New York.

When any variable (e.g., aryl, heterocycle, R², etc.) occurs more than one time in any constituent or in Formula (I), its definition on each occurrence is independent of its definition at every other occurrence.

As used herein, the term “composition” is intended to encompass a product comprising the specified ingredients in the specified amounts, as well as any product which results, directly or indirectly, from combination of the specified ingredients in the specified amounts.

Prodrugs and solvates of the compounds of the invention are also contemplated herein. The term “prodrug”, as employed herein, denotes a compound that is a drug precursor which, upon administration to a subject, undergoes chemical conversion by metabolic or chemical processes to yield a compound of Formula I or a salt and/or solvate thereof. A discussion of prodrugs is provided in T. Higuchi and V. Stella, Pro-drugs as Novel Delivery Systems (1987) 14 of the A.C.S. Symposium Series, and in Bioreversible Carriers in Drug Design, (1987) Edward B. Roche, ed. American Pharmaceutical Association and Pergamon Press, both of which are incorporated herein by reference thereto.

“Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate” is a solvate wherein the solvent molecule is H₂O.

“Effective amount” or “therapeutically effective amount” as used herein, refers to an amount of Compound of Formula (I) and/or an additional therapeutic agent, or a composition thereof that is effective in producing the desired therapeutic, ameliorative, inhibitory or preventative effect when administered to a patient suffering from a Condition, In the combination therapies of the present invention, an effective amount can refer to each individual agent or to the combination as a whole, wherein the amounts of all agents administered are together effective, but wherein the component agent of the combination may not be present individually in an effective amount.

The compounds of Formula I can form salts which are also within the scope of this invention. Reference to a compound of Formula I herein is understood to include reference to salts thereof, unless otherwise indicated. The term “salt(s)”, as employed herein, denotes acidic salts formed with inorganic and/or organic acids, as well as basic salts formed with inorganic and/or organic bases. In addition, when a compound of Formula I contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. Pharmaceutically acceptable (i.e., non-toxic, physiologically acceptable) salts are preferred, although other salts are also useful. Salts of the compounds of the Formula I may be formed, for example, by reacting a compound of Formula I with an amount of acid or base, such as an equivalent amount, in a medium such as one in which the salt precipitates or in an aqueous medium followed by lyophilization.

Exemplary acid addition salts include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates, naphthalenesulfonates, nitrates, oxalates, phosphates, propionates, salicylates, succinates, sulfates, tartarates, thiocyanates, toluenesulfonates (also known as tosylates,) and the like. Additionally, acids which are generally considered suitable for the formation of pharmaceutically useful salts from basic pharmaceutical compounds are discussed, for example, by P. Stahl at al, Camille G. (eds.) Handbook of Pharmaceutical Salts. Properties, Selection and Use. (2002) Zurich: Wiley-VCH; S. Berge at al, Journal of Pharmaceutical Sciences (1977) 66(1) 1-19; P. Gould, International J. of Pharmaceutics (1986) 33 201-217; Anderson et al, The Practice of Medicinal Chemistry (1996), Academic Press, New York; and in The Orange Book (Food & Drug Administration, Washington, D.C. on their website). These disclosures are incorporated herein by reference thereto.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamines, t-butyl amines, and salts with amino acids such as arginine, lysine and the like. Basic nitrogen-containing groups may be quarternized with agents such as lower alkyl halides (e.g. methyl, ethyl, and butyl chlorides, bromides and iodides), dialkyl sulfates (e.g. dimethyl, diethyl, and dibutyl sulfates), long chain halides (e.g. decyl, lauryl, and stearyl chlorides, bromides and iodides), aralkyl halides (e.g. benzyl and phenethyl bromides), and others.

All such acid salts and base salts are intended to be pharmaceutically acceptable salts within the scope of the invention and all acid and base salts are considered equivalent to the free forms of the corresponding compounds for purposes of the invention.

Pharmaceutically acceptable esters of the present compounds include the following groups: (1) carboxylic acid esters obtained by esterification of the hydroxy groups, in which the non-carbonyl moiety of the carboxylic acid portion of the ester grouping is selected from straight or branched chain alkyl (for example, acetyl, n-propyl, t-butyl, or n-butyl), alkoxyalkyl (for example, methoxymethyl), aralkyl (for example, benzyl), aryloxyalkyl (for example, phenoxymethyl), aryl (for example, phenyl optionally substituted with, for example, halogen, C₁₋₄alkyl, or C₁₋₄alkoxy or amino); (2) sulfonate esters, such as alkyl- or aralkylsulfonyl (for example, methanesulfonyl); (3) amino acid esters (for example, L-valyl or L-isoleucyl); (4) phosphonate esters and (5) mono-, di- or triphosphate esters. The phosphate esters may be further esterified by, for example, a C₁₋₂₀ alcohol or reactive derivative thereof, or by a 2,3-di (C₆₋₂₄)acyl glycerol.

Compounds of Formula I, and salts, solvates and prodrugs thereof, may exist in their tautomeric form (for example, as an amide or imino ether). All such tautomeric forms are contemplated herein as part of the present invention.

All stereoisomers (for example, geometric isomers, optical isomers and the like) of the present compounds (including those of the salts, solvates and prodrugs of the compounds as well as the salts and solvates of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this invention, as are positional isomers (such as, for example, 4-pyridyl and 3-pyridyl). Individual stereoisomers of the compounds of the invention may, for example, be substantially free of other isomers, or may be admixed, for example, as racemates or with all other, or other selected, stereoisomers. The chiral centers of the present invention can have the S or R configuration as defined by the IUPAC 1974 Recommendations. The use of the terms “salt”, “solvate” “prodrug” and the like, is intended to equally apply to the salt, solvate and prodrug of enantiomers, stereoisomers, rotamers, tautomers, positional isomers, racemates or prodrugs of the inventive compounds.

Polymorphic forms of the compounds of Formula I, and of the salts, solvates and prodrugs of the compounds of Formula I, are intended to be included in the present invention.

The compounds according to the invention have pharmacological properties; in particular, the compounds of Formula I are inhibitors of LpxC.

In one aspect, the invention provides a pharmaceutical composition comprising as an active ingredient at least one compound of Formula (I).

In another aspect, the invention provides a pharmaceutical composition of Formula (I) additionally comprising at least one pharmaceutically acceptable carrier.

In another aspect, the invention provides a method of treating disorders associated with LpxC, said method comprising administering to a patient in need of such treatment a pharmaceutical composition, which comprises a therapeutically effective amount of at least one compound of Formula (I).

In another aspect, the invention provides a use of a compound of Formula (I) for the manufacture of a medicament to treat disorders associated with LpxC.

The compounds of Formula I have antibacterial activity and can be useful in the treatment of a microbial infection, including gram negative and gram positive infections.

In another aspect, the invention provides a method of preparing a pharmaceutical composition for treating the disorders associated with LpxC, said method comprising bringing into intimate contact at least one compound of Formula I and at least one pharmaceutically acceptable carrier.

In another aspect, the invention provides a pharmaceutical composition for treating disorders associated with LpxC, in a subject comprising, administering to the subject in need of such treatment a therapeutically effective amount of a compound of Formula I or a pharmaceutically acceptable salt, solvate, ester or isomer thereof.

In another aspect, the invention provides a compound of Formula I in purified form.

In another aspect, the invention provides a method of treating a condition or disease mediated by LpxC (such as a microbial infection), in a subject comprising: administering to the subject in need of such treatment a therapeutically effective amount of at least one compound of Formula I or a pharmaceutically acceptable salt, solvate or isomer thereof.

In another aspect, the invention provides a method for the treatment of a microbial infection in a mammal, comprising administering to said mammal a therapeutically effective amount of a compound of Formula I or a pharmaceutically acceptable salt, solvate or ester thereof.

In one embodiment, the microbe causing the infection is a bacteria, in another embodiment it is a fungus. In one embodiment, the microbial infection is a gram negative infection; in another embodiment, it is a gram positive infection.

In another aspect, the invention provides a method for the treatment of a microbial infection in a mammal, comprising administering to said mammal a therapeutically effective amount of a compound of Formula I in combination with one or more additional antibacterial or antifungal agents. In one embodiment, said additional antibacterial agent is active against gram negative bacteria. In another embodiment, said additional antibacterial agent is active against gram positive bacteria.

In one embodiment, the compounds of Formula (I) can be administered to a subject to treat gram negative bacterial infections. They may also be given along with other antibiotics, such as the macrolides, e.g., erythromycin, rifampicin and azithromycin, to achieve or enhance the gram negative antibacterial activity, or with other non-macrolide antibiotics to achieve or enhance the spectrum or potency of the particular antibacterial agent against gram negative organisms.

Likewise, the compounds of Formula I can be used with other agents, which are in and of themselves useful in conjunction with antibacterial agents. For example, bacterial cell wall permeabilizing agents can be included. Representative examples of such compounds include EDTA, polymixin B nonapeptide, poly-L-lysine and neomycin. Other permeability enhancing agents known to those skilled in the art can be included herein as well.

In another embodiment, the bacterial infection treatable by the compounds of the present invention is caused by at least one organism selected from the group consisting of Acinetobacter baumannii, Acinetobacter calcoaceticus, Acinetobacter haemolyticus, Acinetobacter hydrophila, Actinobacillus actinomycetemcomitans, Aeromonas hydrophila, Alcaligenes xylosoxidans, Bacteroides distasonis, Bacteroides fragilis, Bacteroides melaminogenicus, Bacteroides ovatus, Bacteroides thetaiotaomicron, Bacteroides vulgatus, Bartonella henselae, Bordetella pertussis, Branharmelia catarrhalis Brucella melitensis, Brucella abortus, Brucella canis, Burkholderia cepacia, Burkholderia mallei, Burkholderia pseadomallei, Campylobacter coli, Campylobacter fetus, Campylobacter jejuni, Citrobacter diversus, Citrobacter freundii, Citrobacter koseri, Coxiella burnetli, Edwarsiella tarda, Ehrlichia chafeenis, Eikenella corrondens, Enterobacter aerogenes, Enterobacter agglomerans, Enterobacter cloacae, Escherichia coli, Flavobacterium meningosepticum, Francisella tularensis, Fusobacterium spp., Haemophilus ducreyi, Haemophilus influenzae, Haemophilus parainfluenzae, Helicobacter pylori, Kingella kingae, Klebsiella oxytoca, Klebsiella ozaenae, Klebsiella pneumoniae, Klebsiella rhinoscleromatis, Legionella pneumophila, Moraxella catarrhalis, Morganella morganii, Neisseria gonorrhoeae, Neisseria meningitides, Pasteurella multocida, Plesiomonas shigelloides, Porphyromonas asaccharolytica, Porphyromonas gingivalis, Prevotella bivia, Prevotella buccae, Prevotella corporis, Prevotella endodontalis, Prevotella intermedia, Prevotella melaminogenica, Prevotella oralis, Proteus mirabilis, Proteus myxofaciens, Proteus penner, Proteus vulgaris, Providencia alcalifaciens, Providencia rettgeri, Providencia stuarfii, Pseudomonas aeruginosa, Pseudomonas fluorescens, Ricketsia prowozekii, Salmonella enterica, Serratia marcescens, Shigella boydii, Shigella dysenteriae, Shigella flexneri, Shigella sonnei, Stenotrophomonas maltophilia, Streptobacillus moniliformis, Vibrio alginolyticus, Vibrio cholerae, Vibrio parahaemolyticus, Vibrio vuluificus, Yersinia enterocolitica, Yersinia pestis, and Yersinia pseudotuberculosis.

In another embodiment, the bacterial infection is caused by at least one organism selected from the group consisting of Acinetobacter baumannii, Acinetobacter spp., Aeromonas hydrophila, Bacteroides fragilis, Bacteroides spp., Bordetella pertussis, Campylobacter jejuni, Campylobacter spp., Citrobacter freundii, Citrobacter spp., Enterobacter cloacae, Enterobacter spp., Escherichia coli, Fusobacterium spp., Haemophilus influenzae, Haemophilus parainfluenzae, Helicobacter pylori, Klebsiella pneumoniae, Klebsiella spp., Legionella pneumophila, Moraxella catarrhalis, Morganella morganii, Neisseria gonorrhoeae, Neisseria meningitides, Pasteurella multocida, Prevotella spp., Proteus mirabilis, Proteus spp., Providencia stuartii, Pseudomonas aeruginosa, Pseudomonas spp., Salmonella enterica, Salmonella typhi, Somalia marcescens, Shigella spp., Stenotrophomonas maltophilia, Vibrio cholerae, Vibrio spp., and Yersinia spp.

The standard LpxC assay consists of 0.2 nM LpxC enzyme, 1.0 μM UDP-3-O—(R-3-hydroxymyristoyl)-N-acetylglucosamine, and test compound, in assay buffer and 2% DMSO. Assay buffer is comprised of 25 mM HEPES, pH 7.3, 150 mM NaCl, 2.0 mM DTT, and 0.01% BSA. The enzyme reaction is carried out in a 96-well assay plate, in a final volume of 102 μL. Solutions of test compounds are prepared in 100% DMSO. Reaction additions, in order, are (1) 2.0 μL compound solution, (2) 80 μL of assay buffer, (3) 104 of 10 μM UDP-3-O—(R-3-hydroxymyristoyl)-N-acetylglucosamine (in assay buffer) and, (4) 10 μL of LpxC enzyme (20 nM in assay buffer) to initiate the reaction. In positive control reactions, addition (1) has 2.0 μL of 100% DMSO (without compound); these reactions are used as the total signal (TSB) value. Reactions are incubated at room temperature for 60 minutes when 10 μL of 1 N HCl is added to stop the reaction. The plate is shaken by hand for 10 seconds to ensure complete quenching. Assay plates are sealed with foil tape, and stored at −80° C. for 24-48 hr prior to analysis.

The concentrations of substrate and product in the reaction mixtures are determined with BioTrove's proprietary RapidFire™ high-throughput mass spectrometry (HTMS). Assay mixtures are partially purified with reverse phase chromatography, where they are washed with water containing 5 mM ammonium formate and eluted onto the mass spectrometer in 80% acetonitrile, 20% water, and 5 mM ammonium formate. The mass spectrometry peak areas of the substrate and product are measured to determine the concentration of these analytes. The assay signal is the percentage of substrate that is converted to product. Percent inhibition, % I, in test samples is determined from the following equation:

$$\% I = 100 * \frac{( T S B - SampleSignal )}{( T S B )} .$$

Using this method, the following E. coli IC₅₀ (nM) data were obtained for selected Compounds of Formula (I);

Compounds 11-14, 20, 21, 26, 31, 32, 36, 48-53, 65, 74, 79, 80, 88 and 91 had an IC₅₀ value of less than about 10 μM,

Compounds 11-14, 20, 21, 26, 31, 32, 36, 50, 52, 53, 65, 74, 79, 80, 88 and 91 had an IC₅₀ value of less than about 5 μM.

Compounds 11, 13, 31, 32, 36 and 65 had an IC₅₀ value of less than about

Compounds 13, 31, 32 and 36 had an IC₅₀ value of less than about 0.05 μM.

The pharmaceutical compositions containing the active ingredient may be in a form suitable for oral use, for example, as tablets, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients that are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the technique described in the U.S. Pat. Nos. 4,256,108; 4,166,452; and 4,265,874 to form osmotic therapeutic tablets for controlled release.

Formulations for oral use may also be presented as hard gelatin capsules wherein the active ingredients is mixed with an inert solid diluent, for example, calcium carbonate, calcium phosphate or kaolin, or a soft gelatin capsules where in the active ingredient is mixed with water or an oil medium, for example peanut oil, liquid paraffin or olive oil.

Aqueous suspensions contain the active material in admixture with excipients suitable for the manufacture of aqueous suspensions. Such excipients are suspending agents, for example, sodium carboxymethylcellulose, methylcellulose, hydroxypropylmethyl-cellulose, sodium alginate, polyvinyl-pyrrolidone, gum tragacanth and gum acacia; dispersing or wetting agents may be a naturally-occurring phosphatide, for example, lecithin, or condensation products of an alkylene oxide with fatty acids, for example polyoxyethylene stearate, or condensation products of ethylene oxide with long chain aliphatic alcohols, for example, heptadecaethylene-oxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example, polyethylene sorbitan monooleate. The aqueous suspensions may also contain one or more preservatives, for example, ethyl or n-propyl, p-hydroxybenzoate, one or more coloring agents, one or more flavoring agents, and one or more sweetening agents, such as sucrose, saccharin or aspartame.

Oily suspensions may be formulated by suspending the active ingredient in a vegetable oil, for example, arachis oil, olive oil, sesame oil or coconut oil, or in mineral oil such as liquid paraffin. The oily suspensions may contain a thickening agent, for example, beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, e.g., sweetening, flavoring and coloring agents, may also be present.

The pharmaceutical compositions of the invention may also be in the form of an oil-in-water emulsion. The oily phase may be a vegetable oil, e.g., olive oil or arachis oil, or a mineral oil, e.g., liquid paraffin or mixtures of these. Suitable emulsifying agents may be naturally-occurring phosphatides, e.g., soy beans, lecithin, and esters or partial esters derived from fatty acids and hexitol anhydrides, for example, sorbitan monooleate, and condensation products of the said partial esters with ethylene oxide, e.g., polyoxyethylene sorbitan monooleate. The emulsions may also contain sweetening and flavoring agents.

Syrups and elixirs may be formulated with sweetening agents, for example, glycerol, propylene glycol, sorbitol or sucrose. Such formulations may also contain demulcent, preservative, flavoring and coloring agents.

The pharmaceutical compositions may be in the form of a sterile injectable aqueous or oleagenous suspension. This suspension may be formulated according to the known art using those suitable dispersing or wetting agents and suspending agents, which have been mentioned above. The sterile injectable preparation may also be a sterile injectable solution or suspension in a non-toxic parenterally-acceptable diluent or solvent, e.g., as a solution in 1,3-butane diol. Among the acceptable vehicles and solvents that may be employed are water, Ringer's solution and isotonic sodium chloride solution. In addition, sterile fixed oils are conventionally employed as a solvent or suspending medium. For this purpose any bland fixed oil may be employed including synthetic mono- or diglycerides. In addition, fatty acids such as oleic acid find use in the preparation of injectables.

Compounds of the invention may also be administered in the form of suppositories for rectal administration of the drug. The compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols,

For topical use, creams, ointments, jellies, solutions or suspensions, etc., containing the compound of the invention are employed. (For purposes of this application, topical application shall include mouthwashes and gargles.)

The compounds for the present invention can be administered in the intranasal form via topical use of suitable intranasal vehicles, or via transdermal routes, using those forms of transdermal skin patches well known to those of ordinary skill in the art. To be administered in the form of a transdermal delivery system, the dosage administration will, of course, be continuous rather than intermittent throughout the dosage regimen. Compounds of the present invention may also be delivered as a suppository employing bases such as cocoa butter, glycerinated gelatin, hydrogenated vegetable oils, mixtures of polyethylene glycols of various molecular weights and fatty acid esters of polyethylene glycol.

The dosage regimen utilizing the compounds of the present invention is selected in accordance with a variety of factors including type, species, weight, sex and medical condition of the patient; the severity of the condition to be treated; the route of administration; the renal and hepatic function of the patient; and the particular compound thereof employed. A physician or veterinarian of ordinary skill can readily determine and prescribe the effective amount of the drug required to prevent, counter, arrest or reverse the progress of the condition. Optimal precision in achieving concentration of drug within the range that yields efficacy without toxicity requires a regimen based on the kinetics of the drug's availability to target sites. This involves a consideration of the distribution, equilibrium, and elimination of a drug. Preferably, doses of the compound of Formula I useful in the method of the present invention range from 0.01 to 1000 mg per day. More preferably, dosages range from 0.1 to 1000 mg/day. Most preferably, dosages range from 0.1 to 500 mg/day. For oral administration, the compositions are preferably provided in the form of tablets containing 0.01 to 1000 milligrams of the active ingredient, particularly 0.01, 0.05, 0.1, 0.5, 1.0, 2.5, 5.0, 10.0, 15.0, 25.0, 50.0, 100 and 500 milligrams of the active ingredient for the symptomatic adjustment of the dosage to the patient to be treated. An effective amount of the drug is ordinarily supplied at a dosage level of from about 0.0002 mg/kg to about 50 mg/kg of body weight per day. The range is more particularly from about 0.001 mg/kg to 1 mg/kg of body weight per day.

Advantageously, the active agent of the present invention may be administered in a single daily dose, or the total daily dosage may be administered in dividend doses of two, three or four time daily.

The amount of active ingredient that may be combined with the carrier materials to produce single dosage form will vary depending upon the host treated and the particular mode of administration.

it will be understood, however, that the specific dose level for any particular patient will depend upon a variety of factors including the age, body weight, general health, sex, diet, time of administration, route or administration, rate of excretion, drug combination and the severity of the particular disease undergoing therapy.

The compounds of Formula (I) may be produced by processes known to those skilled in the art and as shown in the following reaction schemes and in the preparations and examples described below. These preparations and examples should not be construed to limit the scope of the disclosure. Alternate mechanistic pathways and analogous structures may be apparent to those skilled in the art. All kinds of isomeric forms of the compounds are considered to be within the scope of this invention.

### EXAMPLES

The following abbreviations are used in the procedures and schemes:
ACN Acetonitrile
AcOH Acetic acid
Anh. Anhydrous
Aq Aqueous
BOC tert-Butoxycarbonyl
° C. degrees Celsius
DCM Dichloromethane
DIEA Diisopropylethylamine
DMF Dimethylformamide
DMSO-d₆ Hexadeuterodimethylsulfoxide
EtOAc Ethyl acetate
HATU O-(7-Azabenzotriazole-1-yl)-N,N,N′N′-tetramethyluronium hexafluorophosphate
HPLC High pressure liquid chromatography
LC-MS Liquid Chromatography Mass Spectrometry
M Molar
MeCN Acetonitrile
MeOH Methanol
min Minutes
mg Milligrams
MHz Megahertz
ml Milliliter
MS Mass Spectroscopy
m/z mass per charge
RT Room temperature
THF Tetrahydrofuran
TLC Thin layer chromatography
t_{R} Retention time
X-Phos 5-Bromo-4-chloro-3-indolyl Phosphate

NMR spectra were acquired on a Mercuryplus 400 MHz NMR Spectrometer (Varian), using CDCl₃ or DMSO-d₆ as solvents. LC-MS data was obtained using an Agilent 1100 Series LC/MSD (quadrupole, API-ES (Atmospheric Pressure Interface Electrospray)) with a capillary voltage set to 3500 V and running in positive mode. Reported analytical HPLC (LC/MS) retention times were obtained using a C18 (150×4.6 mm) reverse-phase column eluting with a 5 or 10 minute gradient of 0.1% is trifluoroacetic acid in water to 95:5 acetonitrile:water at a flow rate of 3 mL/min.

Purification via reverse phase chromatography was accomplished using a C18 reverse phase column with a gradient of 0.1% trifluoroacetic acid in water to 95:5 acetonitrile:water at a flow rate of 20 mL/min. Samples were collected using a UV (Gilson, 254 nm) or mass spectra (Agilent 1100 Series LC/MSD model SL) signal.

Normal phase silica gel chromatography on a Biotage instrument was accomplished using a Quad UV System (P/N 07052) utilizing KP-SIL 32-63 urn columns, 60 Å with flash cartridges 12+M or 25+M.

### Example 1

### Example 1A

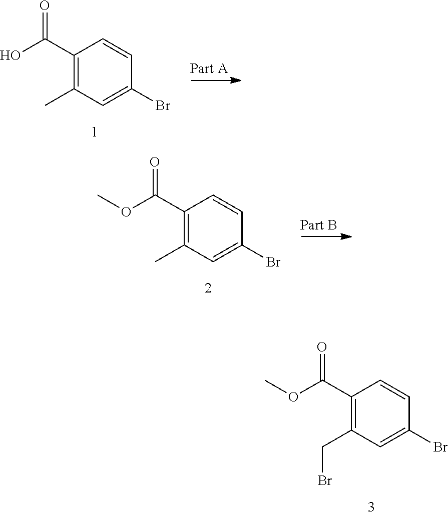

### Part A:

To a solution of 4-bromo-2-methylbenzoic acid (1) (430 mg, 2 mmol) in MeCN (5 mL) and MeOH (5 mL) was added trimethylsilyldiazomethane (2M, 3 mL, 6 mmol). The reaction mixture was stirred at room temperature for 20 minutes, quenched with the addition of AcOH (10%) in MeOH (5 mL), and concentrated to afford crude compound 2. This was further purified by flash column chromatography, gradient elution (0 to 100%) hexane/ethyl acetate, to afford compound 2 as a colorless oil (388 mg, 85% yield).

### Part B:

A solution of compound 2 (388 mg, 1.69 mmol), N-bromosuccinimide (NBS, 302 mg, 1.69 mmol) and benzoyl peroxide (12.3 mg, 0.05 mmol) in carbon tetrachloride (6 mL) was heated to reflux for 18 hours. LC-MS analysis indicated the reaction was complete. The reaction mixture was diluted with diethyl ether (10 mL) and passed through a plug of celite to remove precipitates. The filtrate was washed with saturated NaHCO₃, dried over magnesium sulfate, concentrated and purified by flash column chromatography, gradient elution (0 to 100%) hexane/ethyl acetate, to afford compound 3 as a colorless oil (310 mg, 60% yield). HPLC-MS t_{R}=2.00 min (UV_{254 nm}); mass calculated for formula C₉H₈Br₂O₂ 305.9, observed LCMS m/z 306.9 (M+H).

### Example 1B

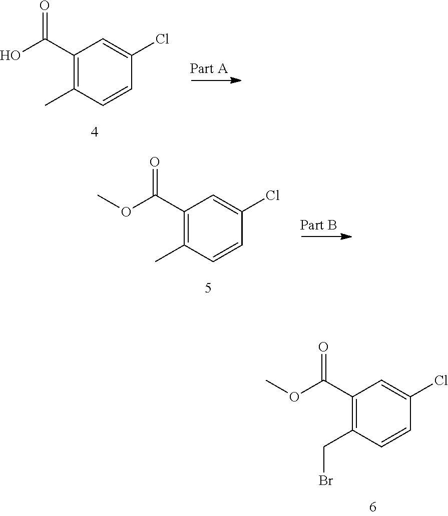

Compound 6 was prepared from 5-chloro-2-methylbenzoic acid (4) using the conditions described in Example 1A, Part A and Part B.

### Example 2

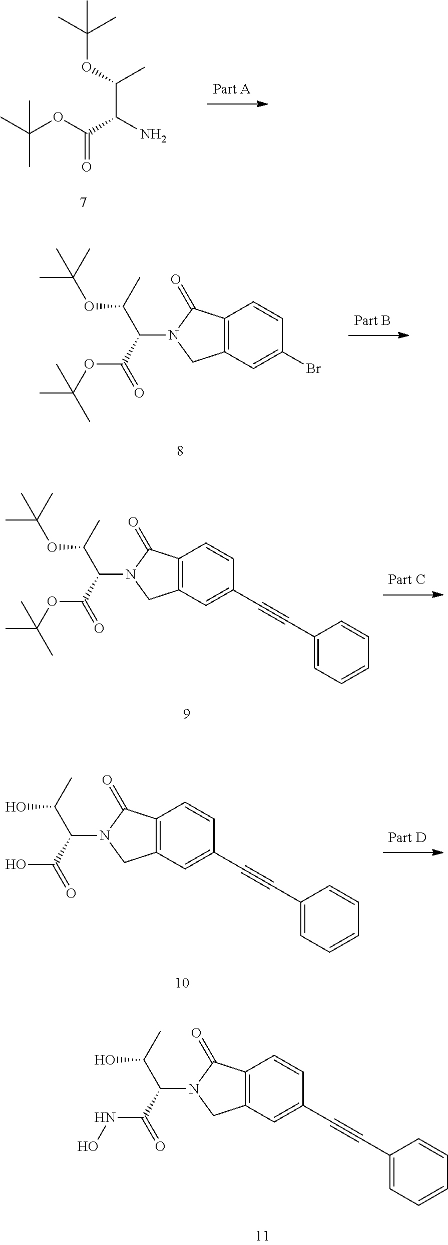

### Part A:

A solution of O-tert-butyl-L-threonine tert-butyl ester hydrochloride (7) (433 mg, 1.62 mmol), compound 3 (550 mg, 1.8 mmol) and DIEA (1.9 mL, 9.72 mmol) in DMF (10 mL) was heated at 80° C. for 18 hours. LC-MS analysis indicated the reaction was complete. The volatiles were removed in vacuo, the residue re-dissolved in EtOAc and washed with 1N HCl. Drying over magnesium sulfate, concentration and purification by flash column chromatography, gradient elution (0 to 100%) hexane/ethyl acetate, afforded compound 8 as a white solid (550 mg, 80% yield). HPLC-MS t_{R}=2.55 min (UV_{254 nm}); mass calculated for formula C₂₀H₂₈BrNO₄ 425.1, observed LCMS m/z 426.1 (M+H).

### Part B:

A solution of compound 8 (143 mg, 0.34 mmol) in acetonitrile (2 mL) was transferred to a Schlenk tube containing dichlorobis(acetonitrile)palladium (II) (0.87 mg, 3.4 mmol), X-Phos (5 mg, 10.2 μmol) and cesium carbonate (285 mg, 0.87 mmol) and the reaction mixture was stirred at room temperature under an inert atmosphere for 25 minutes. 100 μl of a solution containing phenylacetylene (69 mg, 0.67 mmol) in acetonitrile (1 mL) was added and the reaction mixture heated at 90° C. for 15 minutes, The phenylacetylene solution (100 μL) was added every 15 minutes and the reaction mixture was heated at 90° C. for a total of 2.5 hours. LC-MS analysis indicated the reaction was complete, Water (3 mL) was added and the crude product extracted into ethyl acetate (5 mL). Drying over magnesium sulfate, concentration and purification by flash column chromatography, gradient elution (0 to 100%) hexane/ethyl acetate, afforded compound 9 as a yellow solid (130 mg, 87% yield). HPLC-MS t_{R}=2.60 min (UV_{254 nm}); mass calculated for formula C₂₃H₃₃NO₄ 447.2, observed LCMS m/z 448.2 (M+H).

### Part C:

Trifluoroacetic acid (5 mL) was added to compound 9 (50 mg, 0.11 mmol) and the resulting mixture stirred at room temperature for 1 hour, LC-MS analysis indicated hydrolysis was complete. The volatiles were removed in vacuo and the resulting residue re-dissolved in a 1:1 MeCN/water mixture (10 mL) and lyophilized for 18 hours to afford crude compound 10 as a brown solid. HPLC-MS t_{R}=1.65 min (UV_{254 nm}); mass calculated for formula C₂₀H₁₇NO₄ 335.1, observed LCMS m/z 336.1 (M+H).

### Part D:

To a solution of compound 10 (37 mg, 0.11 mmol) and HATU (50 mg, 0.13 mmol) in DMF (2 mL) was added DIEA (57 μL, 0.33 mmol) and O-(tert-butyldimethylsilyl) hydroxylamine (19 mg, 0.13 mmol). The reaction mixture was stirred at room temperature for 18 hours. LC-MS analysis indicated the reaction was complete. The volatiles were removed in vacuo and the resulting residue purified by Prep.HPLC to afford compound 11 (10.5 mg, 28%) as an off white solid.

The compounds in table-1 (11-14) were synthesized using this procedure described in example-2.

TABLE 1

| | | | | Ret. |
|---|---|---|---|---|
| | | Exact | MS m/z | Time |
| Cmpd # | Structure | mass | (M⁺ + H) | (min) |
| 11 | 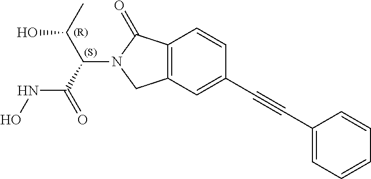 | 350.1 | 351.1 | 3.40 |
| 12 | 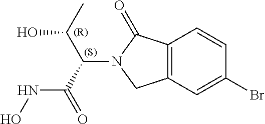 | 328.0 | 329.0 | 2.27 |
| 13 | 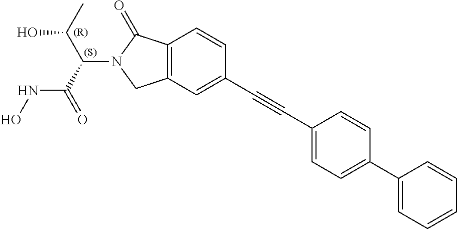 | 426.2 | 427.2 | 4.32 |
| 14 | 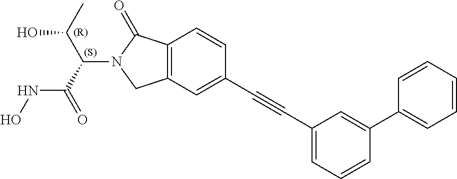 | 426.2 | 427.2 | 4.27 |

### Example 3

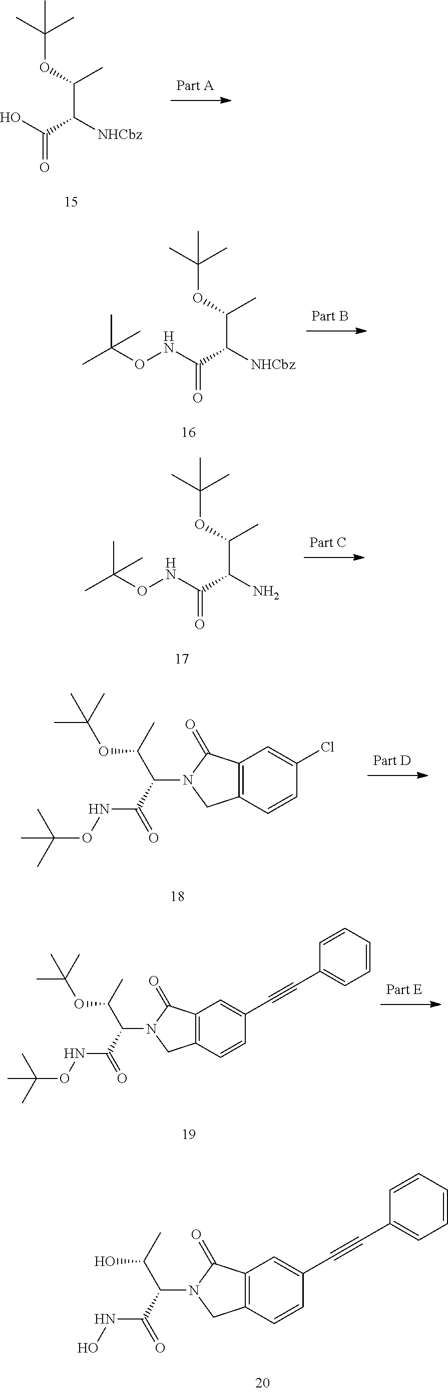

### Part A:

Compound 16 (600 mg, 68%) was prepared from the reaction of N-benzyloxycarbonyl-O-tert-butyl-L-threonine hydrochloride (720 mg, 2.32 mmol) and tert-butyl-O-hydroxylamine (351 mg, 2.78 mmol) using the peptide coupling conditions described in Example 2, Part D. HPLC-MS t_{R}=1.92 min (UV_{254 nm}); mass calculated for formula C₂₀H₃₂N₂O₅ 380.2, observed LCMS m/z 381.2 (M+H).

### Part B:

A solution of compound 16 (600 mg, 1.58 mmol) and palladium on charcoal (10%) in EtOAc (20 mL) was subjected to hydrogenation for 18 hours. LC-MS analysis indicated the reaction was complete. The reaction mixture was filtered by passing through celite, and evaporated to afford crude compound 17 as a white solid (320 mg, 82%). HPLC-MS t_{R}=1.00 min (UV_{254 nm}); mass calculated for formula C₁₂H₂₆N₂O₃ 246.2, observed LCMS m/z 247.3 (M+H).

### Part C:

Compound 18 (120 mg, 66%) was prepared from the reaction of compound 17 (113 mg, 0.46 mmol) and compound 6 (120 mg, 0.46 mmol) using the condensation conditions described in Example 2, Part A. HPLC-MS t_{R}=1.95 min (UV_{254 nm}); mass calculated for formula C₂₀H₂₉ClN₂O₄ 396.2, observed LCMS m/z 397.2 (M+H).

### Part D:

Compound 19 (80 mg, 57%) was prepared from the reaction of compound 18 (120 mg, 0.30 mmol) and phenylacetylene (62 mg, 0.61 mmol) using the Sonagashira coupling conditions described in Example 2, Part B. HPLC-MS t_{R}=2.30 min (UV_{254 nm}): mass calculated for formula C₂₈H₃₄N₂O₄ 462.3, observed LCMS m/z 463.3 (M+H).

### Part E:

Trifluoroacetic acid (2 mL) was added to compound 19 (30 mg, 0.065 mmol) and the resulting mixture stirred at room temperature for 18 hours. LC-MS analysis indicated hydrolysis was complete. The volatiles were removed in vacuo and the resulting residue purified by Prep.HPLC to afford compound 20 (7.8 mg, 35%) as an off white solid.

The compounds 20 and 21 were synthesized using the procedure described in example-3

TABLE 2

| | | | | Ret. |
|---|---|---|---|---|
| | | Exact | MS m/z | Time |
| Cmpd # | Structure | mass | (M⁺ + H) | (min) |
| 20 | 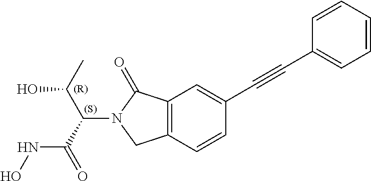 | 350.1 | 351.1 | 3.52 |
| 21 | 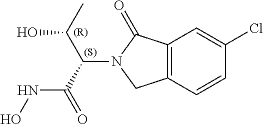 | 284.1 | 285.1 | 2.02 |

### Example 4

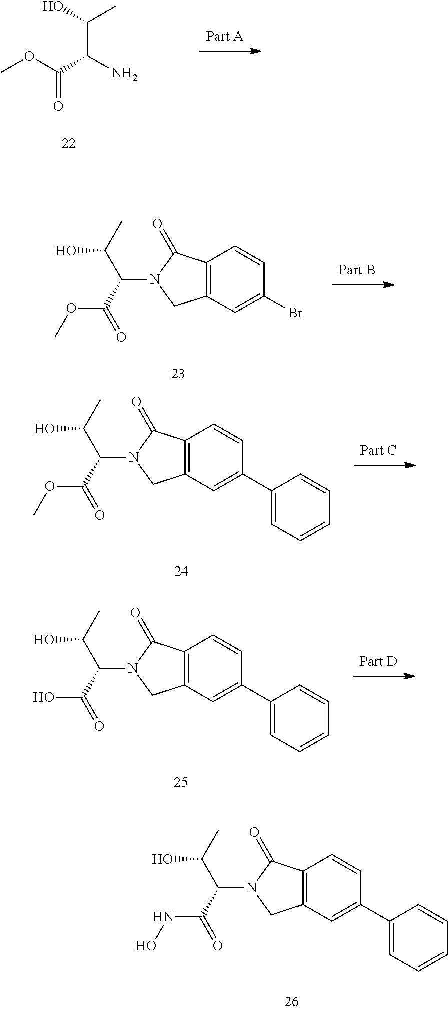

### Part A:

Compound 23 (50 mg, 75%) was prepared from the reaction of threonine methyl ester hydrochloride (35 mg, 0.2 mmol) and compound 6 (100 mg, 0.33 mmol) using the condensation conditions described in Example 2, Part A. HPLC-MS t_{R}=1.45 min (UV_{254 nm}); mass calculated for formula C₁₃H₁₄BrNO₄ 327.0, observed LCMS m/z 328.0 (M+H).

### Part B:

To a mixture of compound 23 (28 mg, 0.086 mmol), potassium phosphate (55 mg, 0.26 mmol) and dichloro[1,1′-bis(diphenylphosphino)ferrocene]palladium (II) dichloromethane adduct (6.3 mg, 8.6 μmol) in dioxane (1 mL) was added phenylboronic acid (12.5 mg, 0.1 mmol). The reaction vessel was flushed with argon, and the reaction mixture heated at 80° C. for 18 hours. LC-MS analysis of the reaction indicated that the reaction was complete. Ethyl acetate (3 mL) was added, and the precipitates removed by passing through a plug of celite. The filtrate was concentrated, and the crude residue purified by flash column chromatography, gradient elution (0 to 100%) hexane/ethyl acetate, to afford compound 24 as a white solid (20 mg, 71% yield). HPLC-MS t_{R}=1.61 min (UV_{254 nm}); mass calculated for formula C₁₉H₁₉NO₄ 325.1, observed LCMS m/z 326.1 (M+H).

### Part C:

A solution containing compound 24 (20 mg, 0.062 mmol) and lithium hydroxide (1M, 68 μL, 0.068 mmol) in THF (2 mL) and water (1 mL) was stirred at room temperature for 1 hour. LC-MS analysis indicated that the hydrolysis was complete. The reaction mixture was acidified to pH 4.0 with 1N HCl, and the crude product extracted into EtOAc (2×10 mL). Drying over magnesium sulfate and concentration afforded compound 25 as a white solid (18 mg, 94%). HPLC-MS t_{R}=1.42 min (UV_{254 nm}); mass calculated for formula C₁₈H₁₇NO₄ 311.1, observed LCMS m/z 312.1 (M+H).

### Part D:

Compound 26 was prepared from compound 25 using the peptide coupling conditions described in Example 2, Part D.

The compound, 26, (Table-3) was synthesized using the procedure described in example 4

TABLE 3

| | | | | Ret. |
|---|---|---|---|---|
| | | Exact | MS m/z | Time |
| Cmpd # | Structure | mass | (M⁺ + H) | (min) |
| 26 | 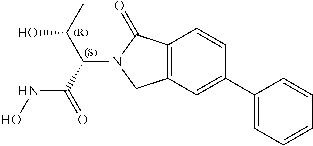 | 326.1 | 327.1 | 2.99 |

### Example 5

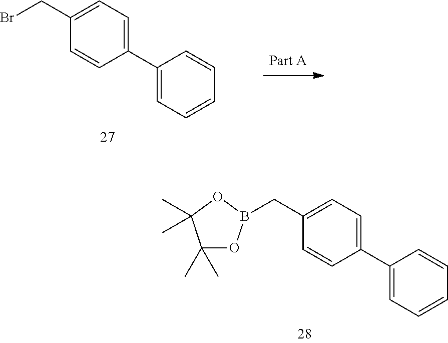

### Part A:

A flask containing a mixture of 4-bromomethylbiphenyl (27) (150 mg, 0.61 mmol), potassium carbonate (252 mg, 1.82 mmol), bis(pinacolato)diboron (185 mg, 0.73 mmol) and tetrakis(triphenylphosphine)palladium (0) (35 mg, 0.03 mmol) in dioxane (3 mL) was flushed with argon, and the reaction mixture heated at 100° C. for 6 hours. LC-MS analysis of the reaction indicated that the reaction was complete. Ethyl acetate (5 mL) was added, and the precipitates removed by passing through a plug of celite. The filtrate was concentrated, and the crude residue purified by flash column chromatography, gradient elution (0 to 100%) hexane/ethyl acetate, to afford compound 28 as a white solid (61 mg, 86% yield). HPLC-MS t_{R}=2.40 min (UV_{254 nm}); mass calculated for formula C₁₉H₂₃BO₂294.2, observed LCMS m/z 295.2 (M+H).

### Example 6

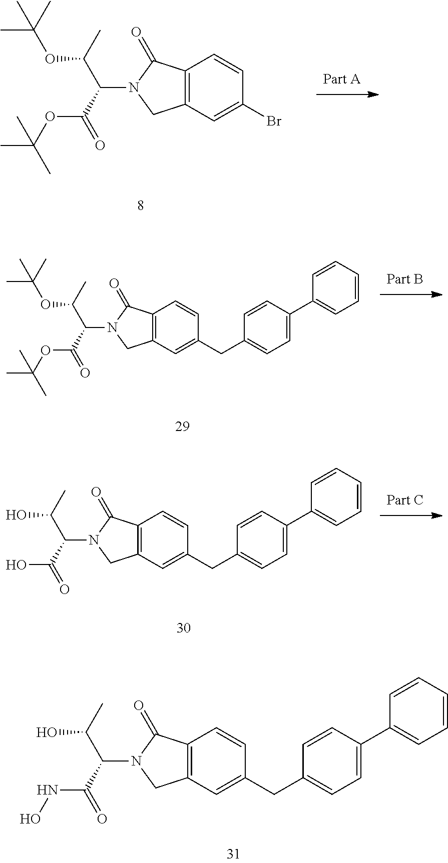

### Part A:

Compound 29 (39 mg, 54%) was prepared from the reaction of compound 8 (60 mg, 0.14 mmol) and compound 28 (83 mg, 0.28 mmol) using the Suzuki coupling conditions described in Example 4, Part B. HPLC-MS t_{R}=2.75 min (UV_{254 nm}); mass calculated for formula C₃₃H₃₉NO₄ 513.3, observed LCMS m/z 514.3 (M+H).

### Part B:

Compound 30 (29 mg, 95%) was prepared from compound 29 (39 mg, 0.076 mmol) using the hydrolysis conditions described in Example 2, Part C. HPLC-MS t_{R}=2.00 min (UV_{254 nm}); mass calculated for formula C₂₅H₂₃NO₄ 401.2, observed LCMS m/z 402.2 (M+H).

### Part C:

Compound 31w as prepared from compound 30 using the peptide coupling conditions described in Example 2, Part D.

The compounds, 31 and 32 (Table-4) were synthesized using the procedure described example-6:

TABLE 4

| | | | | Ret. |
|---|---|---|---|---|
| | | Exact | MS m/z | Time |
| Cmpd # | Structure | mass | (M⁺ + H) | (min) |
| 31 | 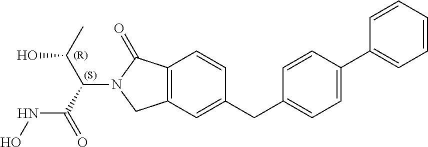 | 416.2 | 417.2 | 4.78 |
| 32 | 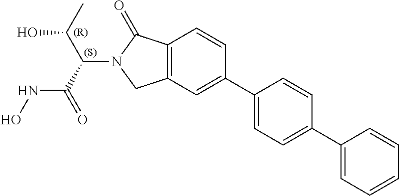 | 402.2 | 403.2 | 4.66 |

### Example 7

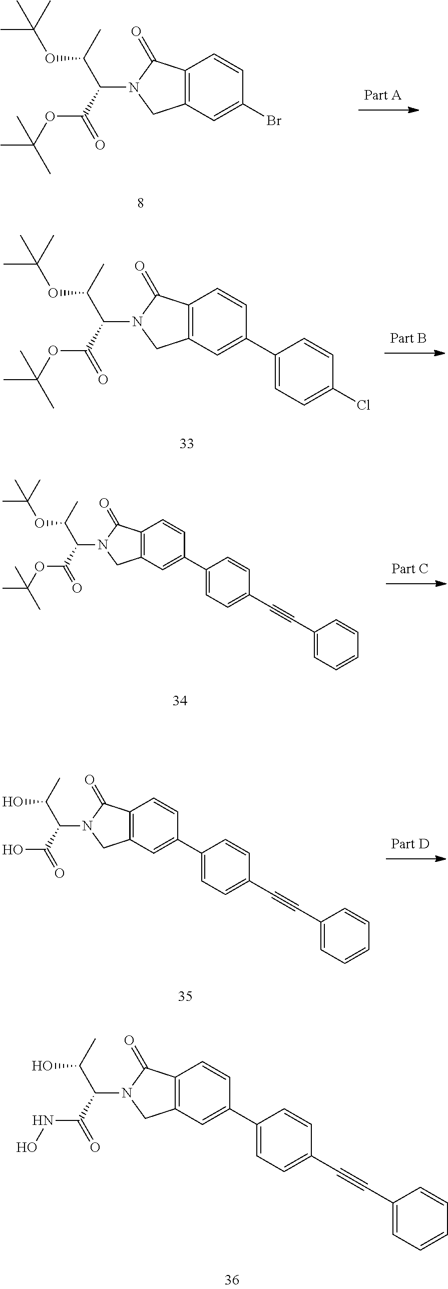

### Part A:

Compound 33 (30 mg, 57 was prepared from the reaction of compound 8 (50 mg, 0.12 mmol) and 4-chlorophenylboronic acid (37 mg, 0.24 mmol) using the Suzuki coupling conditions described in Example 4, Part B. HPLC-MS t_{R}=2.86 min (UV_{254 nm}); mass calculated for formula C₂₆H₃₂ClNO₄ 457.2, observed LCMS m/z 458.2 (M+H).

### Part B:

Compound 34 (20 mg, 59%) was prepared from the reaction of compound 33 (30 mg, 0.065 mmol) and phenylacetylene (13 mg, 0.13 mmol) using the Sonagashira coupling conditions described in Example 2, Part B. HPLC-MS t_{R}=2.66 min (UV_{254 nm}); mass calculated for formula C₃₄H₃₇N₀O₄ 523.3, observed LCMS m/z 524.2 (M+H).

### Part C:

Compound 35 (7.3 mg, 46%) was prepared from compound 34 (20 mg, 0.038 mmol) using the hydrolysis conditions described in Example 2, Part C. HPLC-MS t_{R}=1.97 min (UV_{254 nm}); mass calculated for formula C₂₆H₂₁NO₄ 411.1, observed LCMS m/z 412.1 (M+H).

### Part D:

Compound 36 was prepared from compound 35 using the peptide coupling conditions described in Example 2, Part D.

The compound, 36, was synthesized using the procedure described in example 7

TABLE 5

| | | | | Ret. |
|---|---|---|---|---|
| | | Exact | MS m/z | Time |
| Cmpd # | Structure | mass | (M⁺ + H) | (min) |
| 36 | 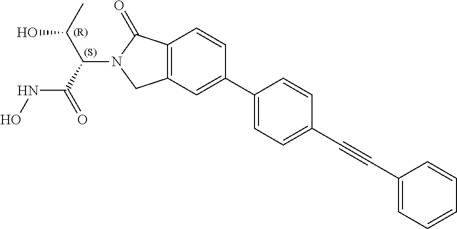 | 426.2 | 427.2 | 4.26 |

### Example 8

### Example 8A

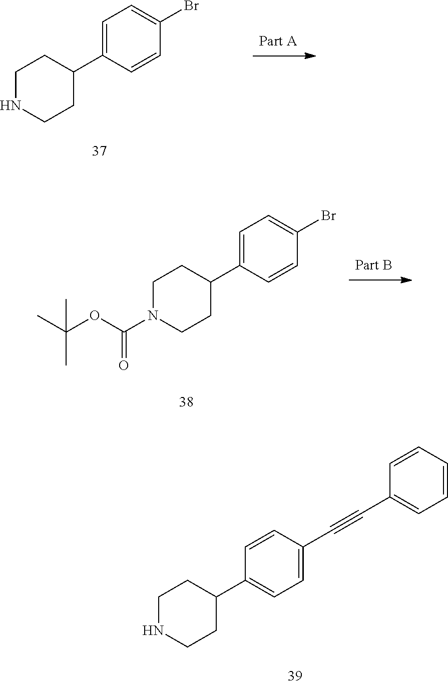

### Part A:

A mixture of 4-(4-bromophenyl)piperidine (37) (960 mg, 4.0 mmol) and di-tert-butyl dicarbonate (960 mg, 4.4 mmol) at 0° C. in DCM (10 mL) was warmed to room temperature and stirred for 3 hours. LC-MS analysis indicated the reaction was complete. Dichloromethane (10 mL) was added and the solution washed with 1N HCl (10 mL). Drying over magnesium sulfate, concentration and purification by flash column chromatography, gradient elution (0 to 100%) hexane/ethyl acetate, afforded compound 38 as a white solid (1.36 g, 100% yield). HPLC-MS t_{R}=2.50 min (UV_{254 nm}); mass calculated for formula C₁₆H₂₂BrNO₂ 339.1, observed LCMS m/z 284.1 (M+H-^{t}Bu).

### Part B:

A solution of compound 38 (600 mg, 1.76 mmol) in acetonitrile (5 mL) was transferred to a Schlenk tube containing dichlorobis(acetonitrile)palladium (II) (4.6 mg, 17.6 □mol), X-Phos (25 mg, 52.9 μmol) and cesium carbonate (1.5 g, 4.59 mmol) and the reaction mixture was stirred at room temperature under an inert atmosphere for 25 minutes. 100 μL of a solution containing phenylacetylene (360 mg, 3.52 mmol) in acetonitrile (2 mL) was added and the reaction mixture heated at 90° C. for 15 minutes. The phenylacetylene solution (100 μL) was added every 15 minutes and the reaction mixture was heated at 90° C. for a total of 2.5 hours. LC-MS analysis indicated the reaction was complete. Water (6 mL) was added and the crude product extracted into ethyl acetate (10 mL). Drying over magnesium sulfate, concentration and purification by flash column chromatography, gradient elution (0 to 100%) hexane/ethyl acetate, afforded BOC-protected compound 39 as a yellow solid (546 mg, 86% yield). HPLC-MS t_{R}=2.70 min (UV_{254 nm}); mass calculated for formula C₂₄H₂₇NO₂ 361.2, observed LCMS m/z 306.2 (M+H-^{t}Bu).

The BOC-protecting group was hydrolyzed by the addition of trifluoroacetic acid (5 mL) and the resulting mixture stirred at room temperature for 1 minute. LC-MS analysis indicated hydrolysis was complete. The volatiles were removed in vacuo and the resulting residue re-dissolved in a 1:1 MeCN/water mixture (10 mL) and lyophilized for 18 hours to afford crude compound 39. HPLC-MS t_{R}=1.22 min (UV_{254 nm}), mass calculated for formula C₁₉H₁₉N 261.2, observed LCMS m/z 262.2 (M+H).

### Example 8B

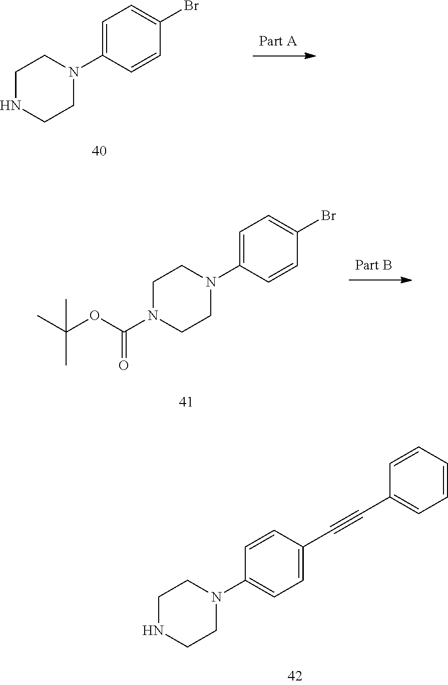

Compound 42 was prepared from 1-(4-bromophenyl)piperazine (40) using the conditions described in Example 8A, Part A and Part B. HPLC-MS t_{R}=1.19 min (UV_{254 nm}); mass calculated for formula C₁₈H₁₈N₂ 262.2, observed LCMS m/z 263.1 (M+H).

### Example 8C

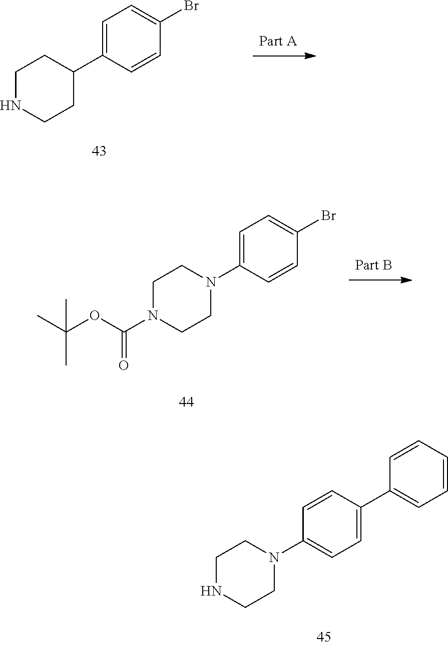

### Part A:

Compound 44 was prepared from 4-(4-bromophenyl)piperidine (43) using the conditions described in Example 8A, Part A. HPLC-MS t_{R}=2.61 min (UV_{254 nm}); mass calculated for formula C₁₆H₂₂BrNO₂ 339.1, observed LCMS m/z 284.0 (M+H-^{t}Bu).

### Part B:

Compound 45 was prepared from the reaction of compound 44 and phenylboronic acid using the Suzuki coupling conditions described in Example 4, Part B. The BOC-protecting group was hydrolyzed by the addition of trifluoroacetic acid (5 mL) and the resulting mixture stirred at room temperature for 1 minute. LC-MS analysis indicated hydrolysis was complete. The volatiles were removed in vacuo and the resulting residue re-dissolved in a 1:1 MeCN/water mixture (10 mL) and lyophilized for 18 hours to afford crude compound 45. HPLC-MS t_{R}=1.24 min (UV_{254 nm}); mass calculated for formula C₁₇H₁₉N 237.2, observed LCMS m/z 238.3 (M+H).

### Example 9

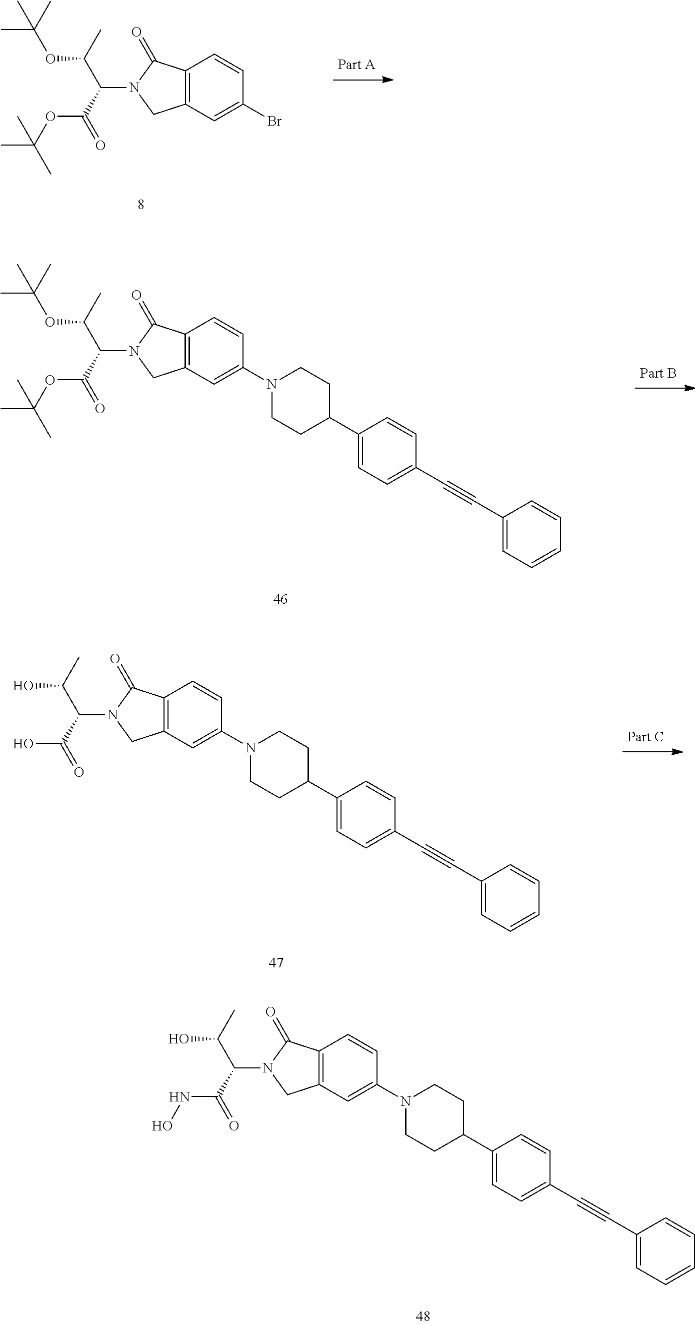

### Part A:

A flask containing a mixture of compound 8 (80 mg, 0.19 mmol), compound 39 (74 mg, 0.28 mmol), potassium phosphate (120 mg, 0.56 mmol), X-Phos (9 mg, 18.8 and tris (dibenzylideneacetone) dipalladium (0) (8.6 mg, 9.4 μmol) in dioxane (3 mL) was flushed with argon, and the reaction mixture heated at 100° C. for 18 hours. LC-MS analysis of the reaction indicated that the reaction was complete. Ethyl acetate (5 mL) was added, and the precipitates removed by passing through a plug of celite. The filtrate was concentrated, and the crude residue purified by flash column chromatography, gradient elution (0 to 100%) hexane/ethyl acetate, to afford compound 46 as a white solid (59 mg, 52% yield). HPLC-MS t_{R}=2.88 min (UV_{254 nm}), mass calculated for formula C₃₉H₄₆N₂O₄ 606.3, observed LCMS m/z 607.3 (M+H).

### Part B:

Compound 47 (42 mg, 88%) was prepared from compound 46 (59 mg, 0.097 mmol) using the hydrolysis conditions described in Example 2, Part C. HPLC-MS t_{R}=214 min (UV_{254 nm}); mass calculated for formula C₃₁H₃₀N₂O₄ 494.2, observed LCMS m/z 495.3 (M+H).

### Part D:

Compound 48 was prepared from compound 47 using the peptide coupling conditions described in Example 2, Part D.

The following compounds, 48 to 53 (Table-6) were synthesized using the procedure described in example-9

TABLE 6

| | | | | Ret. |
|---|---|---|---|---|
| | | Exact | MS m/z | Time |
| Cmpd # | Structure | mass | (M⁺ + H) | (min) |
| 48 | 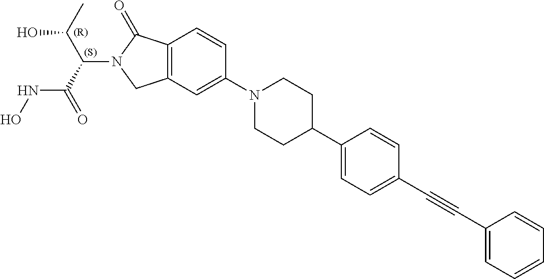 | 509.2 | 510.2 | 5.46 |
| 49 | 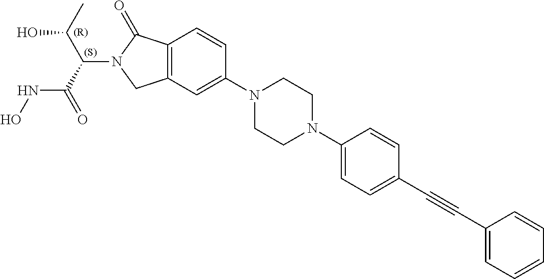 | 510.2 | 511.2 | 4.69 |
| 50 | 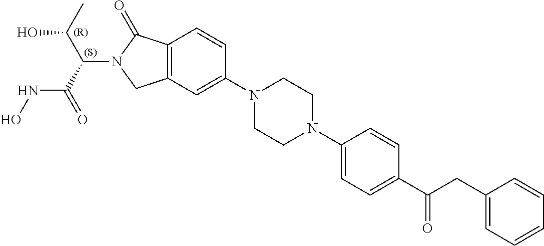 | 528.2 | 529.2 | 4.46 |
| 51 | 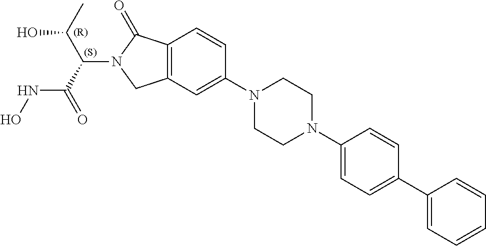 | 486.2 | 487.3 | 4.60 |
| 52 | 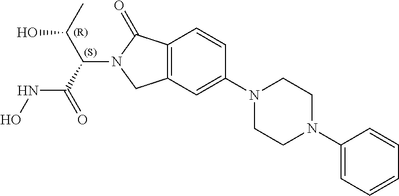 | 410.2 | 411.2 | 2.98 |
| 53 | 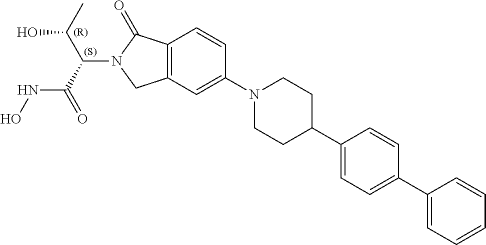 | 485.2 | 486.2 | 4.96 |

### Example 10

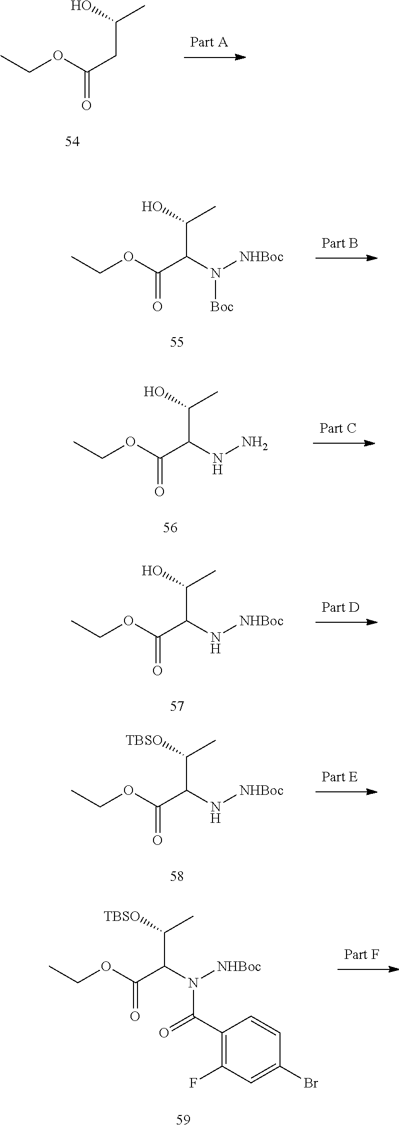

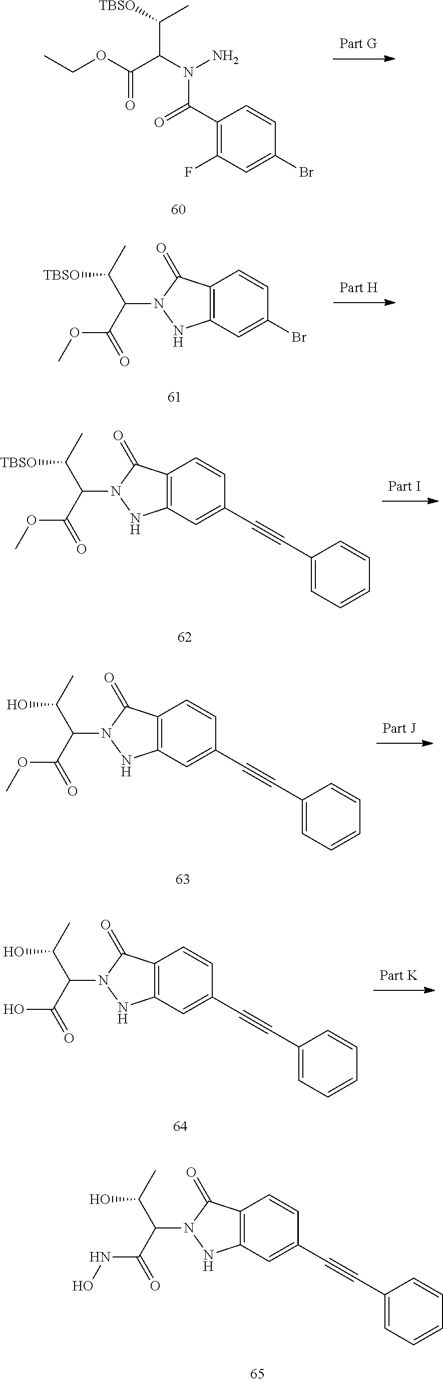

### Part A:

A solution of lithium diisopropylamide (LDA, 1.8M, 14.3 mL, 25.7 mmol) in THF (30 mL) was cooled to −60° C. under an argon atmosphere. (R)-Ethyl 3-hydroxybutanoate (800 μL, 6.11 mmol) was diluted with THF (5 mL) and the resulting solution transferred to the stirring lithium diisopropylamide solution. The reaction mixture was warmed to −20° C. over 30 minutes. The resulting dianion was cooled to −78° C. and a solution of di-tert-butylazodicarboxylate (3.52 g, 15.3 mmol) in THF (7 mL) slowly added. The reaction mixture was stirred at −78° C. for a further 10 minutes before quenching with AcOH (2.1 mL, 36.7 mmol). The reaction mixture was stirred at −78° C. for an additional 15 minutes, warmed to room temperature, diluted with water and extracted with EtOAc. Drying over magnesium sulfate, concentration and purification by flash column chromatography, gradient elution (0 to 100%) hexane/ethyl acetate, afforded the separation of both diastereomers of compound 55 as a colorless oil (2.0 g, 91% yield). Diastereomer 1: HPLC-MS t_{R}=1.94 min (UV_{254 nm}); mass calculated for formula C₁₆H₃₀N₂O₇ 362.2, observed LCMS m/z 385.2 (M+Na). Diastereomer 2: HPLC-MS t_{R}=2.10 min (UV_{254 nm}); mass calculated for formula C₁₆H₃₀N₂O₇ 362.2, observed LCMS m/z 385.2 (M+Na).

### Part B:

Trifluoroacetic acid (2 mL) was added to a stirring solution of compound 55 (500 mg, 1.38 mmol) in DCM (2 mL) and the resulting mixture stirred at room temperature for 30 minutes. LC-MS analysis indicated hydrolysis was complete. The volatiles were removed in vacuo and the resulting residue re-dissolved in a 1:1 MeCN/water mixture (10 mL) and lyophilized for 18 hours to afford crude compound 56. HPLC-MS t_{R} 0.17 min (UV_{254 nm}); mass calculated for formula C₆H₁₄N₂O₃ 162.1, observed LCMS m/z 163.1 (M+H).

### Part C:

Compound 57 was prepared from compound 46 using the conditions described in Example 8A, Part A. Two regioisomers were isolated. Regioisomer 1 (desired) HPLC-MS t_{R}=1.29 min (UV_{254 nm}); mass calculated for formula C₁₁H₂₂N₂O₅ 262.2, observed LCMS m/z 285.2 (M+Na). Regioisomer 2: HPLC-MS t_{R}=1.38 min (UV_{254 nm}); mass calculated for formula C₁₁H₂₂N₂O₅ 262.2, observed LCMS m/z 285.2 (M+Na).

### Part D:

A solution of compound 57 (230 mg, 0.88 mmol), imidazole (174 mg, 2.64 mmol) and tert-butyldimethylsilyl chloride (265 mg, 1.76 mmol) in DMF (10 mL) was stirred at room temperature for 18 hours. LC-MS analysis indicated the reaction was complete. The reaction was quenched with water and extracted with EtOAc. Drying over magnesium sulfate and concentration afforded crude compound 58 as a colorless oil (314 mg, 95%). HPLC-MS t_{R}=2.49 min (UV_{254 nm}); mass calculated for formula C₁₇H₃₆N₂O₅Si 376.2, observed LCMS m/z 321.3 (M+H-^{t}Bu).

### Part E:

### 4-bromo-2-fluorobenzoyl chloride (271 mg, 1.14 mmol) was added to a solution of compound 58 (314 mg, 0.84 mmol) and DIEA (436 μL, 2.5 mmol) in THF (5 mL) and the reaction mixture heated at 60° C. for 1 hour. LC-MS analysis of the reaction indicated that the reaction was complete. The reaction was quenched with the addition of 1N HCl and extracted with EtOAc. Drying over magnesium sulfate and concentration afforded crude compound 59 which was further purified by flash column chromatography, gradient elution (0 to 100%) hexane/ethyl acetate (320 mg, 66%). HPLC-MS t_{R}=2.80 min (UV_{254 mm}); mass calculated for formula C₂₄H₃₈BrFN₂O₄Si 576.2, observed LCMS m/z 599.2 (M+Na).

### Part F:

Trifluoroacetic acid (2 mL) was added to compound 59 (50 mg, 0.087 mmol) and the resulting mixture stirred at room temperature for 5 minutes. LC-MS analysis indicated BOG-hydrolysis was complete. The volatiles were removed in vacuo and the resulting residue re-dissolved in EtOAc (10 mL) and washed with saturated NaHCO₃. Drying over magnesium sulfate and concentration afforded crude compound 60 (40 mg, 97%) as a white solid. HPLC-MS t=2.57 min (UV₂₅₄); mass calculated for formula C₁₉H₃₀BrFN₂O₄Si 476.2, observed LCMS 477.2 (M+H).

### Part G:

A solution of compound 60 (40 mg, 0.084 mmol) in DMF (5 mL) was heated at 130° C. for 7 hours. LC-MS analysis of the reaction confirmed product formation but also hydrolysis of the tert-butyldimethylsilyl protecting group. Imidazole (17 mg, 0.25 mmol) and tert-butyldimethylsilyl chloride (25 mg, 0.17 mmol) was added and the reaction mixture stirred at room temperature for 18 hours. Water (10 mL) was added and the crude product extracted with EtOAc (2×10 mL). Drying over magnesium sulfate and concentration afforded crude compound 61 which was further purified by flash column chromatography, gradient elution (0 to 100%) hexane/ethyl acetate (25 mg, 66%). HPLC-MS t_{R}=2.30 min (UV_{254 nm}); mass calculated for formula C₁₉H₂₉BrN₂O₄Si 456.1, observed LCMS m/z 457.1 (M+H).

### Part H:

Compound 62 was prepared from the reaction of compound 61 (40 mg, 0.09 mmol) with phenyl acetylene (18 mg, 0.18 mmol) using the Sonagashira coupling conditions described in Example 8A, Part B. HPLC-MS t_{R}=2.61 min (UV_{254 nm}); mass calculated for formula C₂₇H₃₄N₂O₄Si 478.3, observed LCMS m/z 479.2 (M+H).

### Part I:

A solution containing compound 62 (21.5 mg, 0.045 mmol) and tetrabutylammonium fluoride (1M, 45 μL, 0.045 mmol) in THF (2 mL) was stirred at room temperature for 1 hour. LC-MS analysis indicated that the hydrolysis was complete. The reaction mixture was quenched with the addition of saturated NH₄Cl and extracted with EtOAc (2×5 mL). Drying over magnesium sulfate and concentration afforded crude compound 63 which was subjected to flash silica chromatography, gradient elution (0 to 20%) ethyl acetate/methanol (10 mg, 61%). HPLC-MS t_{R}=1.89 min (UV_{254 nm}): mass calculated for formula C₂₁H₂₀N₂O₄ 364.1, observed LCMS m/z 365.2 (M+H).

### Part J:

Compound 64 (9 mg, 100%) was prepared from compound 63 (10 mg, 0.027 mmol) using the saponification conditions described in Example 4, Part C. HPLC-MS t_{R}=1.49 min (UV_{254 nm}); mass calculated for formula C₁₉H₁₆N₂O₄ 336.1, observed LCMS m/z 337.1 (M+H).

### Part K:

Compound 65 was prepared from compound 64 using the peptide coupling conditions described in Example 2, Part D.

The following compound, 65 was synthesized using the procedure described 10

TABLE 7

| | | | | Ret. |
|---|---|---|---|---|
| | | Exact | MS m/z | Time |
| Cmpd # | Structure | mass | (M⁺ + H) | (min) |
| 65 | 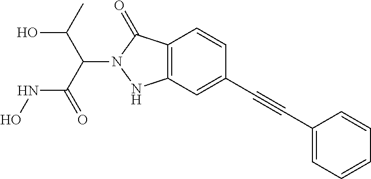 | 351.1 | 352.1 | 3.19 |

### Example 11

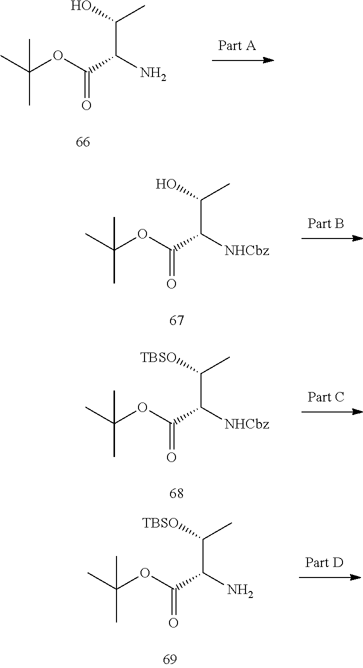

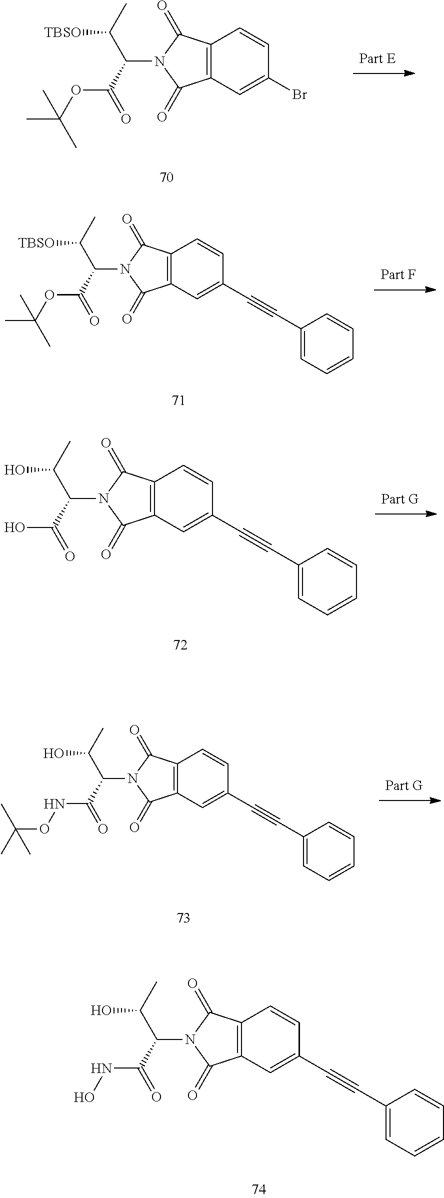

### Part A:

To an ice-cooled solution of L-threonine tert-butyl ester hydrochloride (66) (2.12 g, 10 mmol) and DIEA (3.83 mL, 22 mmol) in THF (20 mL) was slowly added over 5 minutes a solution of benzyl chloroformate (1.55 mL, 11 mmol) in THF (10 mL). The reaction mixture was warmed to room temperature and stirred for 2 hours. LC-MS analysis indicated the reaction was complete. The reaction was quenched with the addition of 1N HCl and extracted with EtOAc. Drying over magnesium sulfate and concentration afforded crude compound 67 as a yellow oil (2.67 g, 100%).

### Part B:

A solution of compound 67 (653 mg, 2.1 mmol), imidazole (173 mg, 2.5 mmol) and tert-butyldimethylsilyl chloride (350 mg, 2.3 mmol) in DMF (10 mL) was stirred at room temperature for 18 hours. LC-MS analysis indicated the reaction was complete. The reaction was quenched with water and extracted with EtOAc. Drying over magnesium sulfate and concentration afforded crude compound 68 as a colorless oil (738 mg, 83%). HPLC-MS t_{R}=2.73 min (UV_{254 nm}); mass calculated for formula C₂₂H₃₇NO₅Si 423.2, observed LCMS m/z 446.1 (M+Na).

### Part C:

A solution of compound 68 (738 mg, 1.74 mmol) and palladium on charcoal (10%) in EtOAc (20 mL) was subjected to hydrogenation for 18 hours. LC-MS analysis indicated the reaction was complete. The reaction mixture was filtered by passing through celite, and evaporated to afford crude compound 69 as a colorless oil (488 mg, 97%). HPLC-MS t_{R}=1.36 min (UV_{254 nm}); mass calculated for formula C₁₄H₃₁NO₃Si 289.2, observed LCMS m/z 290.3 (M+H).

### Part D:

A mixture of compound 69 (145 mg, 0.5 mmol), DIEA (174 □L, 1.0 mmol) and 4-bromophthalic anhydride (170 mg, 0.75 mmol) in dioxane (2 mL) was heated in the microwave for 10 minutes at 160° C. LC-MS analysis indicated the reaction was complete. The volatiles were removed in vacuo and the crude residue subjected to flash silica chromatography, gradient elution (0 to 100%) hexane/ethyl acetate to afford compound 70 as a colorless oil (101 mg, 41%). HPLC-MS t_{R}=2.76 min (UV_{254 nm}); mass calculated for formula C₂₂H₃₂BrNO₅Si 497.1, observed LCMS m/z 498.1 (M+H).

### Part E:

Compound 71 (50 mg, 48%) was prepared from the reaction of compound 70 (100 mg, 0.2 mmol) with phenylacetylene (41 mg, 0.4 mmol) using the Sonagashira coupling conditions described in Example 8A, Part B. HPLC-MS t_{R}=2.94 min (UV_{254 nm}); mass calculated for formula C₃₀H₃₇NO₅Si 519.2. observed LCMS m/z 464.2 (M+H-^{t}Bu).

### Part F:

Trifluoroacetic acid (2 mL) was added to a stirring solution of compound 71 (71 mg, 0.096 mmol) in DCM (2 mL) and the resulting mixture stirred at room temperature for 10 minutes. LC-MS analysis indicated hydrolysis was complete. The volatiles were removed in vacuo and the resulting residue re-dissolved in a 1:1 MeCN/water mixture (10 mL) and lyophilized for 18 hours to afford crude compound 72, HPLC-MS t_{R}=1.72 min (UV_{254 nm}): mass calculated for formula C₂₀H₁₅NO₅ 349.1, observed LCMS m/z 350.1 (M÷H).

### Part G:

Compound 73 was prepared from the reaction of compound 72 (15 mg, 0.043 mmol) and tert-butyl-O-hydroxylamine (11 mg, 0.086 mmol) using the peptide coupling conditions described in Example 2, Part D. HPLC-MS t_{R}=2.0 min (UV_{254 nm}); mass calculated for formula C₂₄H₂₄N₂O₅ 420.2, observed LCMS m/z 421.2 (M+11).

### Part H:

Trifluoroacetic acid (2 mL) was added to compound 73 (10 mg, 0.024 mmol) and the resulting mixture stirred at room temperature for 8 hours. LC-MS analysis indicated hydrolysis was complete. The volatiles were removed in vacuo and the resulting residue purified by Prep.HPLC to afford compound 74 (1.2 mg, 14%) as an off white solid.

The following compound, 74 was synthesized using the procedure described in example 11

TABLE 8

| | | | | Ret. |
|---|---|---|---|---|
| | | Exact | MS m/z | Time |
| Cmpd # | Structure | mass | (M⁺ + H) | (min) |
| 74 | 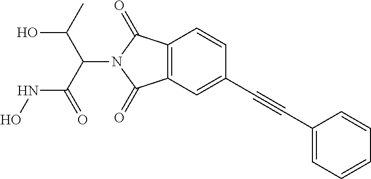 | 364.1 | 365.1 | 3.70 |

### Example 12

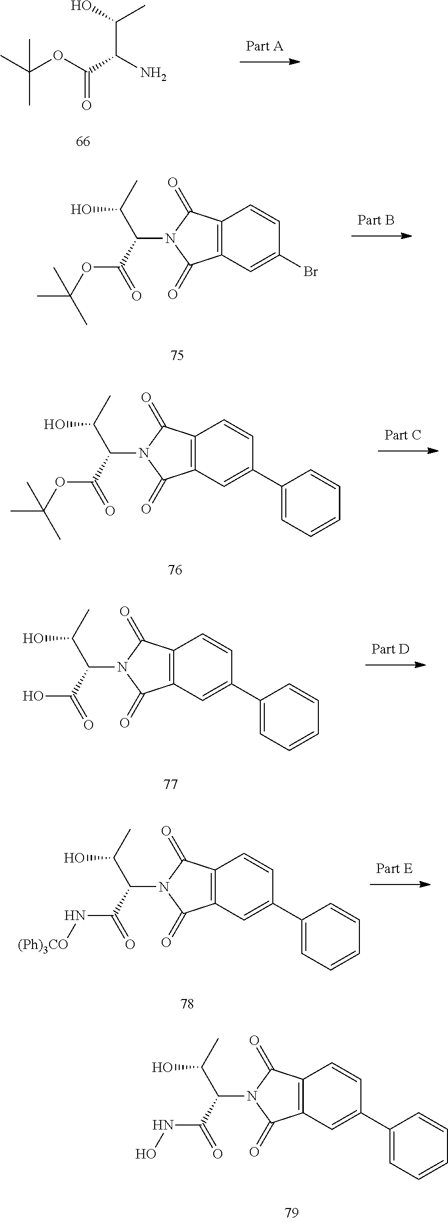

### Part A:

Compound 75 (160 mg, 42%) was prepared from the reaction of L-threonine Pert-butyl ester hydrochloride (66) (211 mg, 1 mmol) and 4-bromophthalic anhydride (341 mg, 1.5 mmol) using the condensation conditions described in Example 11, Part D. HPLC-MS t_{R}=1.85 min (UV_{254 nm}); mass calculated for formula C₁₆H₁₈BrNO₅ 383.0, observed LCMS m/z 384.0 (M+H).

### Part B:

Compound 76 (40 mg, 70%) was prepared from the reaction of compound 75 (56 mg, 0.15 mmol) and phenylboronic acid (21 mg, 0.18 mmol) using the Suzuki coupling conditions described in Example 4, Part B. HPLC-MS t_{R}=2.15 min (UV_{254 nm}); mass calculated for formula C₂₂H₂₃NO₅ 381.2, observed LCMS m/z 326.2 (M+H-^{t}Bu).

### Part C:

Compound 77 (75 mg, 100%) was prepared from compound 76 (90 mg, 0.23 mmol) using the hydrolysis conditions described in Example 11, Part F. HPLC-MS t_{R}=1.24 min (UV_{254 nm}); mass calculated for formula C₁₂H₁₀BrNO₅ 327.0, observed LCMS m/z 328.0 (M+H).

### Part D:

Compound 78 was prepared from the reaction of compound 77 (40 mg, 0.12 mmol) and trityl-O-hydroxylamine (41 mg, 0.14 mmol) using the peptide coupling conditions described in Example 2, Part D. HPLC-MS t_{R}=2.49 min (UV_{254 nm}); mass calculated for formula C₃₇H₃₀N₂O₅ 582.2, observed LCMS m/z 583.2 (M+H).

### Part H:

Trifluoroacetic acid (2 mL) was added to compound 78 (15 mg, 0.025 mmol) and the resulting mixture stirred at room temperature for 1 minute. LC-MS analysis indicated hydrolysis was complete. The volatiles were removed in vacuo and the resulting residue purified by Prep.HPLC to afford compound 79 (1.8 mg, 21%) as an off white solid.

The following compounds 79 and 80 (Table-9) were synthesized using this procedure example 12

TABLE 9

| | | | | Ret. |
|---|---|---|---|---|
| | | Exact | MS m/z | Time |
| Cmpd # | Structure | mass | (M⁺ + H) | (min) |
| 79 | 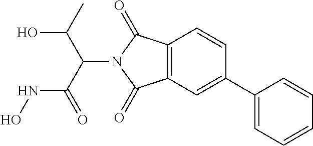 | 340.1 | 341.1 | 3.11 |
| 80 | 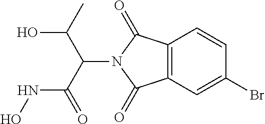 | 342.0 | 343.0 | 2.56 |

### Example 13

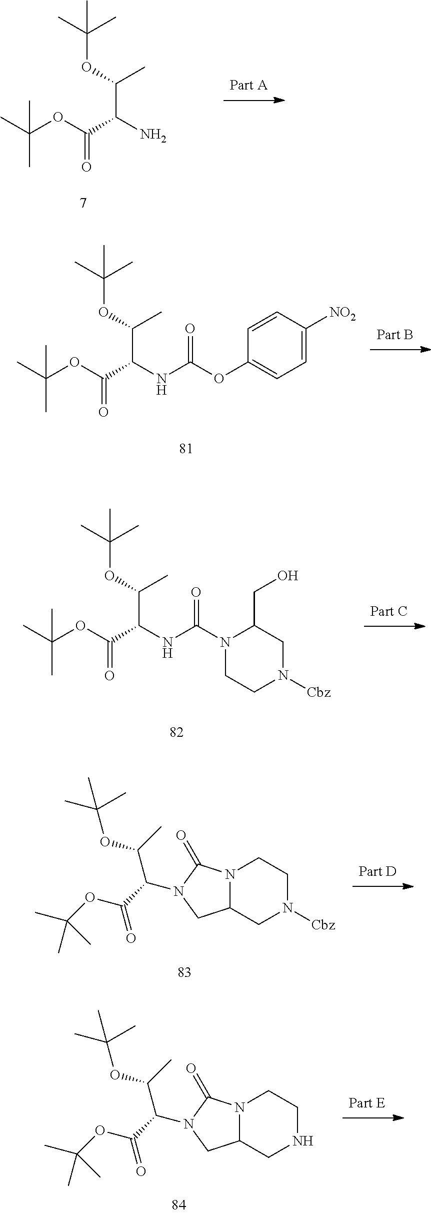

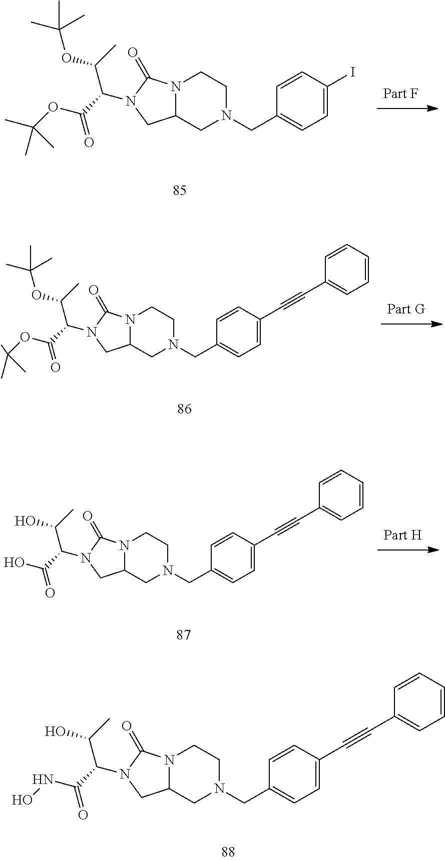

### Part A:

To an ice-cooled solution of 4-nitrophenyl chloroformate (665 mg, 3.3 mmol) and DIEA (1.6 mL, 9 mmol) in THF (10 mL) was slowly added over 20 minutes a solution of O-tert-butyl-L-threonine tert-butyl ester hydrochloride (7) (803 mg, 3 mmol) in THF (5 mL). The reaction mixture was warmed to room temperature and stirred for 18 hours. LC-MS analysis indicated the reaction was complete. The reaction was quenched with the addition of saturated NaHCO₃ and extracted with EtOAc. Drying over magnesium sulfate and concentration afforded compound 81 which was subjected to flash silica chromatography, gradient elution (0 to 100%) hexane/ethyl acetate (917 mg, 77%). HPLC-MS t_{R}=2.22 min (UV_{254 nm}), mass calculated for formula C₁₉H₂₈N₂O₇ 396.2, observed LCMS m/z 397.1 (M+H).

### Part B:

To a solution of compound 81 (396 mg, 1.0 mmol) and DIEA (0.523 □L, 3.0 mmol) in THF (10 mL) was added 4-N-benzyloxycarbonyl-2-hydroxymethylpiperazine (300 mg, 1.2 mmol) and the reaction mixture heated at 80° C. for 18 hours. The reaction was quenched with the addition of 1N HCl and extracted with EtOAc. Drying over magnesium sulfate and concentration afforded compound 82 which was subjected to flash silica chromatography, gradient elution (0 to 100%) hexane ethyl acetate (345 mg, 68 a/0). HPLC-MS t_{R}=1.99 min (UV_{254 nm}); mass calculated for formula C₂₆H₄₁N₃O₇ 507.3, observed LCMS m/z 508.3 (M+H),

### Part C:

Methanesulfonyl chloride (61 μL, 0.79 mmol) was added to an ice-cooled solution of compound 82 (334 mg, 0.66 mmol) in DCM (6 mL) and pyridine (3 mL). The reaction mixture was stirred at 0° C. for 1 hour and then warmed to room temperature. LC-MS analysis indicated the reaction was complete, The reaction was quenched with the addition of 1N HCl and extracted with EtOAc. Drying over magnesium sulfate and concentration afforded compound 83 which was subjected to flash silica chromatography, gradient elution (0 to 100%) hexane/ethyl acetate (290 mg, 90%), HPLC-MS t_{R}=1.39 min (UV_{254 em}); mass calculated for formula C₂₆H₃₉N₃O₆ 489.3, observed LCMS m/z 490.3 (M+H).

### Part D:

Compound 84 (210 mg, 100%) was prepared from compound 73 (290 mg, 0.59 mmol) using the hydrogenation conditions described in Example 3, Part B. HPLC-MS t_{R}=1.19 min (UV_{254 nm}); mass calculated formula C₁₈H₃₃N₃O₄ 355.2, observed LCMS m/z 356.3 (M+H),

### Part E:

A mixture of compound 84 (112 mg, 0.32 mmol), potassium carbonate (52 mg, 0.38 mmol) and 4-iodobenzyl bromide (112 mg, 038 mmol) in DMF (5 mL) was heated at 60° C. for 3 hours, LC-MS analysis indicated the reaction was complete. The reaction was quenched with the addition of saturated NaHCO₃ and extracted with EtOAc.

Drying over magnesium sulfate and concentration afforded compound 85 which was subjected to flash silica chromatography, gradient elution (0 to 100%) hexane/ethyl acetate (25 mg, 14%). HPLC-MS t_{R}=1.56 min (UV_{254 nm}); mass calculated for formula C₂₅H₃₈IN₃O₄ 571.2, observed LCMS m/z 572.2 (M+H).

### Part F:

To a mixture of compound 85 (25 mg, 0.043 mmol), copper iodide (0.5 mg, 2.58 μmol) and dichlorobis(triphenylphosphine)palladium (II) (1.1 mg, 1.5 μmol) in THF (2 mL) was added phenylacetylene (7 mg, 0.065 mmol) and triethylamine (14 μL, 0.1 mmol). The reaction vessel was flushed with argon, and the reaction mixture stirred at room temperature for 18 hours. LC-MS analysis of the reaction indicated that the reaction was complete. Ethyl acetate (5 mL) was added and the reaction mixture washed with saturated NaHCO₃. Drying over magnesium sulfate, concentration and purification by flash column chromatography, gradient elution (0 to 100%) hexane/ethyl acetate, afforded compound 86 as a yellow solid (23 mg, 98% yield). HPLC-MS t_{R}=1.97 min (UV_{254 nm}); mass calculated for formula C₃₃H₄₃N₃O₄ 545.3, observed LCMS m/z 546.3 (M+H).

### Part G:

Trifluoroacetic acid (3 mL) was added to compound 86 (23 mg, 0.42 mmol) and the resulting mixture stirred at room temperature for 1 hour. LC-MS analysis indicated hydrolysis was complete. The volatiles were removed in vacuo and the resulting residue re-dissolved in a 1:1 MeCN/water mixture (10 mL) and lyophilized for 18 hours to afford crude compound 87. HPLC-MS t_{R}=1.50 min (UV_{254 nm}); mass calculated for formula C₂₅H₂₇N₃O₄ 433.2, observed LCMS m/z 434.2 (M+H).

### Part H:

Compound 88 was prepared from compound 87 using the peptide coupling conditions described in Example 2, Part D.

The following compound, 88 was synthesized using the procedure described in example 13:

TABLE 10

| | | | | Ret. |
|---|---|---|---|---|
| | | Exact | MS m/z | Time |
| Compound # | Structure | mass | (M⁺ + H) | (min) |
| 88 | 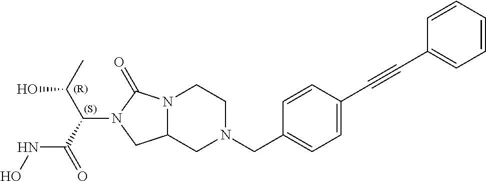 | 448.2 | 449.2 | 2.93 |

### Example 14

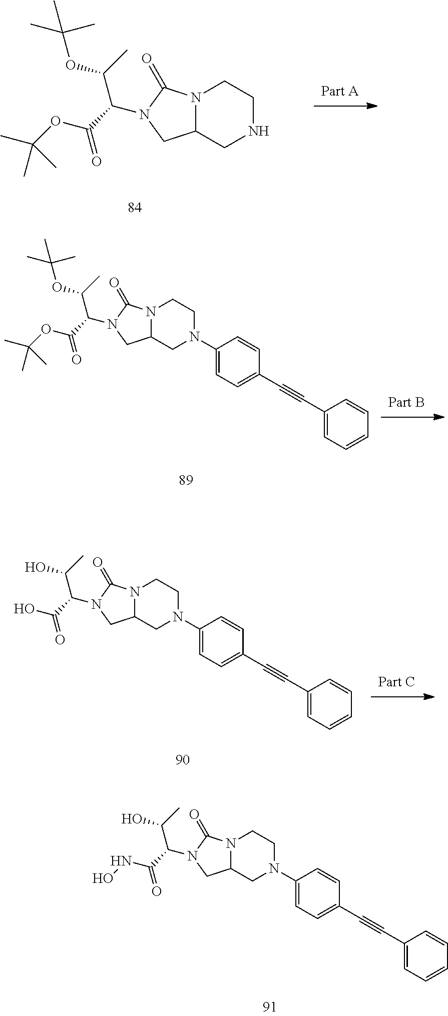

### Part A:

Compound 89 (40 mg, 23%) was prepared from the reaction of compound 84 (118 mg, 0.33 mmol) and 1-bromo-4-(phenylethynyl)benzene using the coupling conditions described in Example 9, Part A. HPLC-MS t_{R}=1.89 min (UV_{254 nm}), mass calculated for formula C₃₂H₄₁N₃O₄ 531.3, observed LCMS m/z 532.3 (M+H).

### Part B:

Trifluoroacetic acid (3 mL) was added to compound 89 (25 mg, 0.047 mmol) and the resulting mixture stirred at room temperature for 1 hour. LC-MS analysis indicated hydrolysis was compete. The volatiles were removed in vacuo and the resulting residue re-dissolved in a 1:1 MeCN/water mixture (10 mL) and lyophilized for 18 hours to afford crude compound 90. HPLC-MS t_{R}=1.55 min (UV_{254 nm}); mass calculated for formula C₂₄H₂₅N₃O₄ 419.2, observed LCMS m/z 420.2 (M+H).

### Part H:

Compound 91 was prepared from compound 90 using the peptide coupling conditions described in Example 2, Part D.

The following compound, 91 was synthesized using the procedure in example 14:

TABLE 11

| | | | | Ret. |
|---|---|---|---|---|
| | | Exact | MS m/z | Time |
| Compound # | Structure | mass | (M⁺ + H) | (min) |
| 91 | 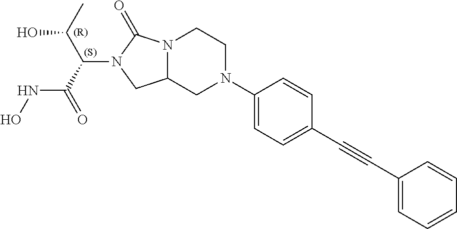 | 434.2 | 435.2 | 3.44 |

It will be appreciated by those skilled in the art that changes could be made to the embodiments described above without departing from the broad inventive concept thereof. It is understood, therefore, that this invention is not limited to the particular embodiments disclosed, but it is intended to cover modifications that are within the spirit and scope of the invention, as defined by the appended claims.

Each and every document referred to in this patent application is incorporated herein by reference in its entirety for all purposes.

1. A compound having the formula: 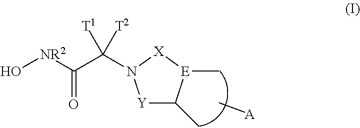
and pharmaceutically acceptable salts, solvates and esters thereof, wherein:
T¹ and T² are each independently selected from the group consisting of H, alkyl, alkenyl, alkynyl, aryl, cycloalkenyl, cycloalkyl, aralkyl, aralkenyl, aralkenyl, cycloalkenylalkyl, cycloalkenylalkenyl, cyclylalkyl, cyclylalkenyl, heteroaryl, heterocyclenyl, heterocyclyl, heteroaralkyl, heteroaralkenyl, heterocyclenylalkyl, heterocyclenylalkenyl, heterocyclylalkyl, and heterocyclylalkenyl, wherein each of said alkyl, alkenyl, alkynyl, aryl, cycloalkenyl, cycloalkyl, aralkyl, aralkenyl, aralkenyl, cycloalkenylalkyl, cycloalkenylalkenyl, cyclylalkyl, cyclylalkenyl, heteroaryl, heterocyclenyl, heterocyclyl, heteroaralkyl, heteroaralkenyl, heterocyclenylalkyl, heterocyclenylalkenyl, heterocyclylalkyl, and heterocyclylalkenyl can be unsubstituted or substituted with one or more moieties independently selected from the group consisting of H, alkyl, alkenyl, alkynyl, aryl, cycloalkenyl, cycloalkyl, aralkyl, aralkenyl, cycloalkenylalkyl, cycloalkenylalkenyl, cycloalkylalkyl, cycloalkylalkenyl, —OH, O-aryl, O-cycloalkenyl, —O-cycloalkyl, —O-heteroaryl, —O-heterocyclenyl, —O-heterocyclyl, —O-alkyl, —O-alkenyl, —O-alkynyl, —O-aralkyl, —O-aralkenyl, —O-cycloalkenylalkyl, —O-cycloalkylalkyl, —SH, S-aryl, S-cycloalkenyl, —S-cycloalkyl, —S-heteroaryl, —S-heterocyclenyl, —S-heterocyclyl, —S-alkyl, —S-alkenyl, —S-alkynyl, —S-aralkyl, —S-aralkenyl, —S-cycloalkenylalkyl, —S-cycloalkylalkyl, —NH₂, NH-aryl, NH-cycloalkenyl, —NH-cycloalkyl, —NH-heteroaryl, —NH-heterocyclenyl, —NH-heterocyclyl, —NH-alkyl, —NH-alkenyl, —NH-alkynyl, —NH-aralkyl, —NH-aralkenyl, —NH-cycloalkenylalkyl, —NH-cycloalkylalky, further wherein each of said alkyl, alkenyl, alkynyl, aryl, cycloalkenyl, cycloalkyl, aralkyl, aralkenyl, aralkenyl, cycloalkenylalkyl, cyclylalkyl, heteroaryl, heterocyclenyl, heterocyclyl, heteroaralkyl, heteroaralkenyl, heterocyclenylalkyl, and heterocyclylalkyl can be unsubstituted or independently substituted from the group consisting of H, alkyl, alkenyl, alkynyl, aryl, cycloalkenyl, cycloalkyl, aralkyl, aralkenyl, cycloalkenylalkyl, and cycloalkylalkyl;
or
T¹ and T² together with the carbon to which they are attached form a ring selected from the group consisting of spirocyclyl, spirocycloalkenyl, spiroheterocyclyl, and spiroheterocyclenyl, wherein each of said spirocyclyl, spirocycloalkenyl, spiroheterocyclyl, and spiroheterocyclenyl can be unsubstituted or substituted with up to three moieties, independently selected from the group consisting of H, alkyl, alkenyl, alkynyl, aryl, cycloalkenyl, cycloalkyl, aralkyl, aralkenyl, cycloalkenylalkyl, cycloalkylalkyl, —OH, O-aryl, O-cycloalkenyl, —O-cycloalkyl, —O-heteroaryl, —O-heterocyclenyl, —O-heterocyclyl, —O-alkyl, —O-alkenyl, —O-alkynyl, —O-aralkyl, —O-aralkenyl, —O-cycloalkenylalkyl, —O-cycloalkylalkyl, —SH, S-aryl, S-cycloalkenyl, —S-cycloalkyl, —S-heteroaryl, —S-heterocyclenyl, —S-heterocyclyl, —S-alkyl, —S-alkenyl, —S-alkynyl, —S-aralkyl, —S-aralkenyl, —S-cycloalkenylalkyl, —S-cycloalkylalkyl, —NH₂, —NH-aryl, —NH-cycloalkenyl, —NH-cycloalkyl, —NH-heteroaryl, —NH-heterocyclenyl, —NH-heterocyclyl, —NH-alkyl, —NH-alkenyl, —NH-alkynyl, —NH-aralkyl, —NH-aralkenyl, —NH-cycloalkenylalkyl, and —NH-cycloalkylalkyl;
X and Y are independently selected from the group consisting of —CR⁵R⁶, —C(O), —S(O)₂, —C(NH)— and NR⁵, wherein each of R⁵ and R⁶ is independently selected from the group consisting of H, alkyl, alkenyl, alkynyl, aryl, cycloalkenyl, cycloalkyl, heteroaryl, heterocyclenyl, heterocyclyl, aralkyl, aralkenyl, cycloalkenylalkyl, cycloalkylalkyl, heteroaralkyl, heterocyclenylalkyl and heterocyclylalkyl, where in each of said alkyl, alkenyl, alkynyl, aryl, cycloalkenyl, cycloalkyl, heteroaryl, heterocyclenyl, heterocyclyl, aralkyl, aralkenyl, cycloalkenylalkyl, cycloalkylalkyl, heteroaralkyl, heterocyclenylalkyl and heterocyclylalkyl can be unsubstituted or substituted with up to three moieties independently selected from the group consisting of H, alkyl, alkenyl, alkynyl, aryl, cycloalkenyl, cycloalkyl, aralkyl, aralkenyl, cycloalkenylalkyl, and cycloalkylalkyl;
E is C, CH, or N; 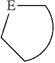
is a six-membered ring selected from the group consisting of aryl, cycloalkenyl, cycloalkyl, heteroaryl, heterocyclenyl and heterocyclyl;
A is selected from the group consisting of alkyl, alkenyl, alkynyl, cycloalkyl, cycloalkenyl, spirocyclyl, spirocycloalkenyl, spiroheterocyclyl, spiroheterocyclenyl, aryl, aralkyl, aralkenyl, cycloalkenylalkyl, cycloalkylalkyl, heteroaryl, heterocyclenyl, heterocyclyl, heteroaralkyl, heterocyclenylalkyl, heterocyclylalkyl, halo, —C(O)R, —C(S)R and —C(NH)R, wherein each of said alkyl, alkenyl, alkynyl, cycloalkyl, cycloalkenyl, spirocyclyl, spirocycloalkenyl, spiroheterocyclyl, spiroheterocyclenyl, aryl, aralkyl, aralkenyl, cycloalkenylalkyl, cycloalkylalkyl, heteroaryl, heterocyclenyl, heterocyclyl, heteroaralkyl, heterocyclenylalkyl, heterocyclylalkyl, is unsubstituted or substituted with up to three moieties independently selected from the group consisting of halo, —OH, O-aryl, O-cycloalkenyl, —O-cycloalkyl, —O-heteroaryl, —O-heterocyclenyl, —O-heterocyclyl, —O-alkyl, —O-alkenyl, —O-alkynyl, —O-aralkyl, —O-aralkenyl, —O-cycloalkenylalkyl, —O-cycloalkylalkyl, —SH, S-aryl, S-cycloalkenyl, —S-cycloalkyl, —S-heteroaryl, —S-heterocyclenyl, —S-heterocyclyl, —S-alkyl, —S-alkenyl, —S-alkynyl, —S-aralkyl, —S-aralkenyl, —S-cycloalkenylalkyl, —S-cycloalkylalkyl, —NH₂, —NH-aryl, —NH-cycloalkenyl, —NH-cycloalkyl, —NH-heteroaryl, —NH-heterocyclenyl, —NH-heterocyclyl, —NH-alkyl, —NH-alkenyl, —NH-alkynyl, —NH-aralkyl, —NH-aralkenyl, —NH-cycloalkenylalkyl, —NH-cycloalkylalkyl, aryl, cycloalkenyl, cycloalkyl, heteroaryl, heterocyclenyl, heterocyclyl, alkyl, alkenyl and alkynyl, wherein each of said aryl, cycloalkenyl, cycloalkyl, heteroaryl, heterocyclenyl, heterocyclyl, alkyl, alkenyl, alkynyl, O-aryl, O-cycloalkenyl, —O-cycloalkyl, —O-heteroaryl, —O-heterocyclenyl, —O-heterocyclyl, —O-alkyl, —O-alkenyl, —O-alkynyl, —O-aralkyl, —O-aralkenyl, —O-cycloalkenylalkyl, —O-cycloalkylalkyl, S-aryl, S-cycloalkenyl, —S-cycloalkyl, —S-heteroaryl, —S-heterocyclenyl, —S-heterocyclyl, —S-alkyl, —S-alkenyl, —S-alkynyl, —S-aralkyl, —S-aralkenyl, —S-cycloalkenylalkyl, —S-cycloalkylalkyl, —NH-aryl, —NH-cycloalkenyl, —NH-cycloalkyl, —NH-heteroaryl, —NH-heterocyclenyl, —NH-heterocyclyl, —NH-alkyl, —NH-alkenyl, —NH-alkynyl, —NH-aralkyl, —NH-aralkenyl, —NH-cycloalkenylalkyl, —NH-cycloalkylalkyl, aryl, cycloalkenyl, cycloalkyl, heteroaryl, heterocyclenyl, heterocyclyl, alkyl, alkenyl, alkynyl can be unsubstituted or substituted with up to three moieties independently selected from the group consisting of halo, —OH, O-aryl, O-cycloalkenyl, —O-cycloalkyl, —O-heteroaryl, —O-heterocyclenyl, —O-heterocyclyl, —O-alkyl, —O-alkenyl, —O-alkynyl, —O-aralkyl, —O-aralkenyl, —O-cycloalkenylalkyl, —O-cycloalkylalkyl, —SH, S-aryl, S-cycloalkenyl, —S-cycloalkyl, —S-heteroaryl, —S-heterocyclenyl, —S-heterocyclyl, —S-alkyl, —S-alkenyl, —S-alkynyl, —S-aralkyl, —S-aralkenyl, —S-cycloalkenylalkyl, and —S-cycloalkylalkyl, —NH₂, —NH-aryl, —NH-cycloalkenyl, —NH-cycloalkyl, —NH-heteroaryl, —NH-heterocyclenyl, —NH-heterocyclyl, —NH-alkyl, —NH-alkenyl, —NH-alkynyl, —NH-aralkyl, —NH-aralkenyl, —NH-cycloalkenylalkyl, —NH-cycloalkylalkyl, aryl, cycloalkenyl, cycloalkyl, heteroaryl, heterocyclenyl, heterocyclyl, alkyl, alkenyl, alkynyl, wherein each of said O-aryl, O-cycloalkenyl, —O-cycloalkyl, —O-heteroaryl, —O-heterocyclenyl, —O-heterocyclyl, —O-alkyl, —O-alkenyl, —O-alkynyl, —O-aralkyl, —O-aralkenyl, —O-cycloalkenylalkyl, —O-cycloalkylalkyl, S-aryl, S-cycloalkenyl, —S-cycloalkyl, —S-heteroaryl, —S-heterocyclenyl, —S-heterocyclyl, —S-alkyl, —S-alkenyl, —S-alkynyl, —S-aralkyl, —S-aralkenyl, —S-cycloalkenylalkyl, —S-cycloalkylalkyl, —NH-aryl, —NH-cycloalkenyl, —NH-cycloalkyl, —NH-heteroaryl, —NH-heterocyclenyl, —NH-heterocyclyl, —NH-alkyl, —NH-alkenyl, —NH-alkynyl, —NH-aralkyl, —NH-aralkenyl, —NH-cycloalkenylalkyl, —NH-cycloalkylalkyl, aryl, cycloalkenyl, cycloalkyl, heteroaryl, heterocyclenyl, heterocyclyl, alkyl, alkenyl and alkynyl can be unsubstituted or substituted with one or more moieties independently selected from the group consisting of halo, —OH, O-aryl, O-cycloalkenyl, —O-cycloalkyl, —O-heteroaryl, —O-heterocyclenyl, —O-heterocyclyl, —O-alkyl, —O-alkenyl, —O-alkynyl, —O-aralkyl, —O-aralkenyl, —O-cycloalkenylalkyl, —O-cycloalkylalkyl, —SH, S-aryl, S-cycloalkenyl, —S-cycloalkyl, —S-heteroaryl, —S-heterocyclenyl, —S-heterocyclyl, —S-alkyl, —S-alkenyl, —S-alkynyl, —S-aralkyl, —S-aralkenyl, —S-cycloalkenylalkyl, —S-cycloalkylalkyl, —NH-aryl, —NH-cycloalkenyl, —NH-cycloalkyl, —NH-heteroaryl, —NH-heterocyclenyl, —NH-heterocyclyl, —NH-alkyl, —NH-alkenyl, —NH-alkynyl, —NH-aralkyl, —NH-aralkenyl, —NH-cycloalkenylalkyl, —NH-cycloalkylalkyl, aryl, cycloalkenyl, cycloalkyl, heteroaryl, heterocyclenyl, heterocyclyl, alkyl, alkenyl and alkynyl;
R² is independently selected from the group consisting of H, alkyl, alkenyl, alkynyl, cycloalkyl, cycloalkenyl, aryl, heteroaryl, heterocyclyl and heterocyclenyl, wherein each of said alkyl, alkenyl, alkynyl, cycloalkyl, cycloalkenyl, aryl, heteroaryl, heterocyclyl, and heterocyclenyl can be unsubstituted or substituted with up to three moieties independently selected from the group consisting of halogen, alkyl, alkenyl, alkynyl, aryl, cycloalkenyl, cycloalkyl, aralkyl, aralkenyl, cycloalkenylalkyl, cycloalkylalkyl, —OH, O-aryl, O-cycloalkenyl, —O-cycloalkyl, —O-heteroaryl, —O-heterocyclenyl, —O-heterocyclyl, —O-alkyl, —O-alkenyl, —O-alkynyl, —O-aralkyl, —O-aralkenyl, —O-cycloalkenylalkyl, —O-cycloalkylalkyl, —SH, S-aryl, S-cycloalkenyl, —S-cycloalkyl, —S-heteroaryl, —S-heterocyclenyl, —S-heterocyclyl, —S-alkyl, —S-alkenyl, —S-alkynyl, —S-aralkyl, —S-aralkenyl, —S-cycloalkenylalkyl, —S-cycloalkylalkyl, —NH₂, —NH-aryl, —NH-cycloalkenyl, —NH-cycloalkyl, —NH-heteroaryl, —NH-heterocyclenyl, —NH-heterocyclyl, —NH-alkyl, —NH-alkenyl, —NH-alkynyl, —NH-aralkyl, —NH-aralkenyl, —NH-cycloalkenylalkyl, and —NH-cycloalkylalky.

2. The compound of claim 1, wherein T¹ is H and T² is ethyl or isopropyl, wherein said ethyl or isopropyl is substituted with —OH or NH₂.

3. The compound according to claim 1, wherein T¹ and T² together with the carbon to which they are attached form a ring form selected from the group consisting of spirocyclyl, spirocycloalkenyl, spiroheterocyclyl, and spiroheterocyclenyl, wherein each of said spirocyclyl, spirocycloalkenyl, spiroheterocyclyl, and spiroheterocyclenyl can be unsubstituted or substituted with one or more moieties independently selected from the group consisting of H, alkyl, alkenyl, alkynyl, aryl, cycloalkenyl, cycloalkyl, aralkyl, aralkenyl, cycloalkenylalkyl, cycloalkylalkyl, —OH, O-aryl, O-cycloalkenyl, —O-cycloalkyl, —O-heteroaryl, —O-heterocyclenyl, —O-heterocyclyl, —O-alkyl, —O-alkenyl, —O-alkynyl, —O-aralkyl, —O-aralkenyl, —O-cycloalkenylalkyl, —O-cycloalkylalkyl, —SH, S-aryl, S-cycloalkenyl, —S-cycloalkyl, —S-heteroaryl, —S-heterocyclenyl, —S-heterocyclyl, —S-alkyl, —S-alkenyl, —S-alkynyl, —S-aralkyl, —S-aralkenyl, —S-cycloalkenylalkyl, and —S-cycloalkylalkyl, —NR″₂, wherein each R″ are independently selected from the group consisting of H, (C₁₋₈)alkyl, (C₁-C₈)alkenyl, alkynyl, aryl, cycloalkenyl, cycloalkyl, aralkyl, aralkenyl, cycloalkenylalkyl, cyclylalkyl, heteroaryl, heterocyclenyl, heterocyclyl, heteroaralkyl, heteroaralkenyl, heterocyclenylalkyl, and heterocyclylalkyl.

4. The compound according to claim 1, wherein said R² is hydrogen.

5. The compound according to claim 1, wherein X is C(O).

6. The compound according to claim 1, wherein Y is C(O) or CH₂.

7. The compound according to claim 1, wherein E is C, CH, or N.

8. The compound according to claim 1, wherein 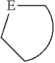
is aryl or heterocyclyl.

9. The compound according to claim 8, wherein said aryl is phenyl or said heterocyclyl is piperazine.

10. The compound of claim 1, wherein A is alkynyl substituted with phenyl, wherein said phenyl can be unsubstituted or further substituted with an additional phenyl.

11. The compound of claim 10, wherein said alkynyl is ethynyl.

12. (canceled)

13. (canceled)

14. The compound of claim 1, wherein A is phenyl, which can be unsubstituted or substituted with alkynyl, wherein said alkynyl is substituted with phenyl.

15. The compound of claim 14, wherein said alkynyl is ethynyl.

16. The compound of claim 1, wherein A is phenyl, substituted with phenyl.

17-22. (canceled)

23. The compound according to claim 1, wherein A is selected from the group consisting of ethynyl, phenyl, phenylmethyl, piperidine, piperazine, bromo and chloro, wherein said ethynyl, phenyl, phenylmethyl, piperidine, and piperazine can be unsubstituted or substituted with an additional phenyl or ethynyl, wherein said additional phenyl can be unsubstituted or substituted with another phenyl or ethynyl, still further wherein, said another phenyl, can be unsubstituted or substituted with still another phenyl.

24. The compound according to claim 1, wherein A is selected from the group consisting of ethynyl, phenyl, phenylmethyl, piperidine, piperazine, bromo and chloro, wherein said ethynyl, phenyl, phenylmethyl, piperidine, and piperazine can be unsubstituted or substituted with an additional phenyl or additional ethynyl, wherein said additional ethynyl is substituted with another phenyl, wherein said another phenyl can be unsubstituted or substituted with still another phenyl.

25. A compound selected from the group consisting of: 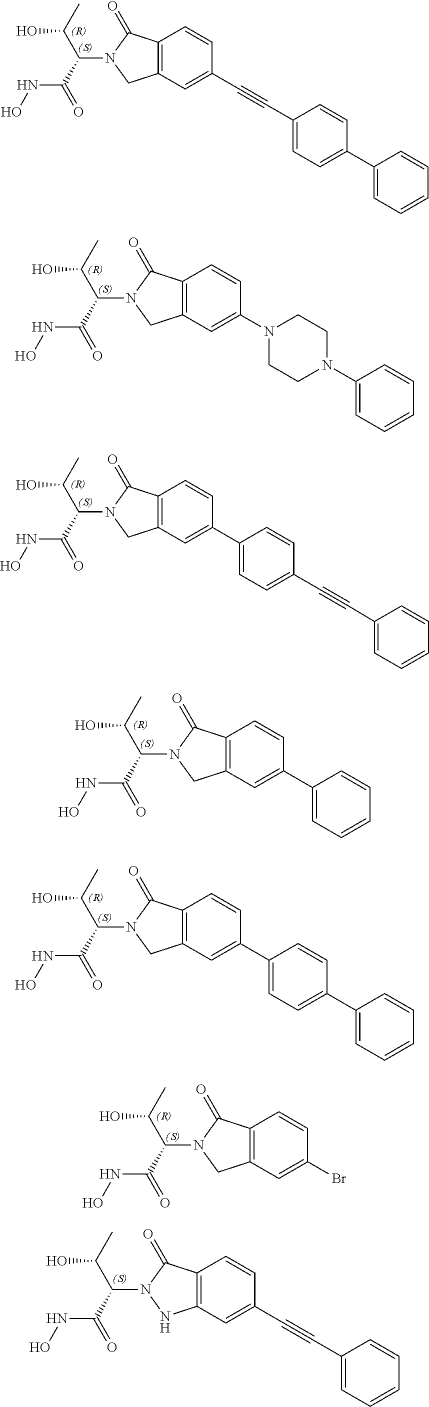 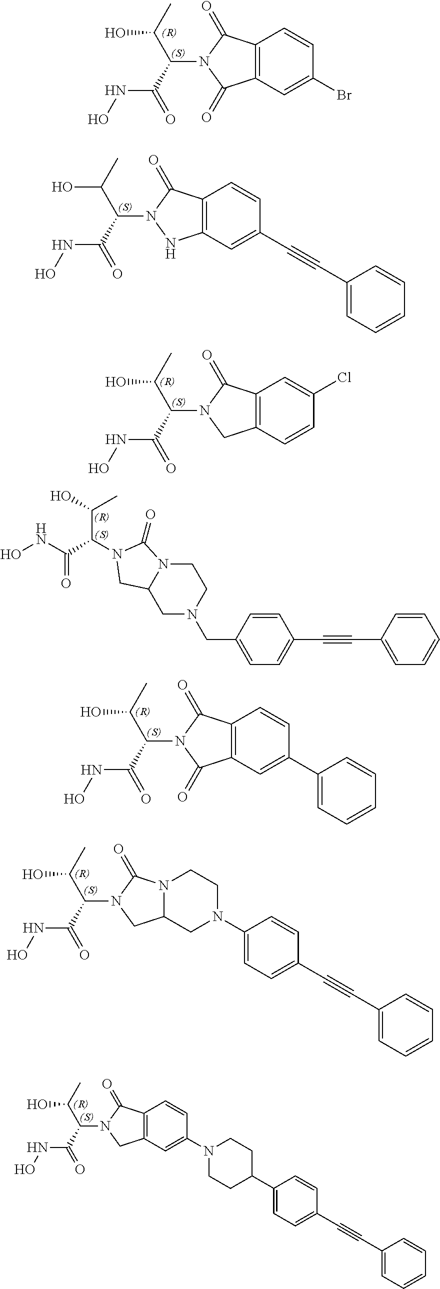 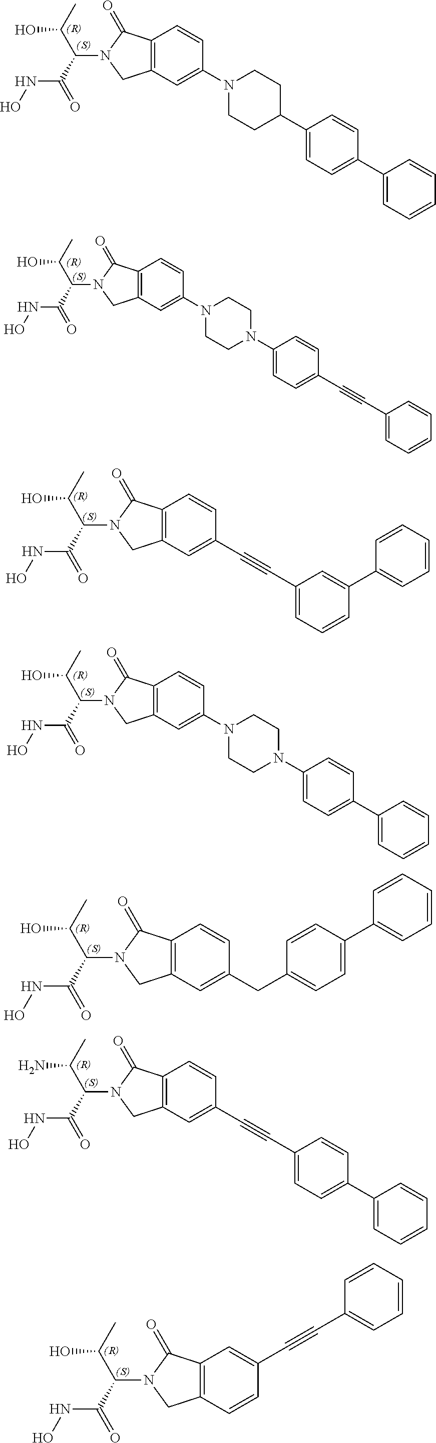 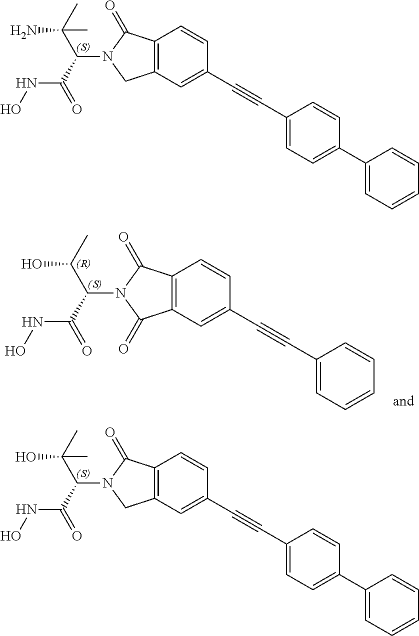
or a pharmaceutically acceptable salt, solvate or ester thereof.

26. (canceled)

27. A pharmaceutical composition comprising at least one compound of claim 1, or a pharmaceutically acceptable salt, solvate or ester thereof, in combination with at least one pharmaceutically acceptable carrier.

28. The pharmaceutical composition of claim 27, further comprising at least one additional agent, drug, medicament, antibody and/or inhibitor for treating a UDP-3-O—(R-3-hydroxymyristoyl)-N-acetylglucosamine deacetylase (LpxC) receptor mediated disease.

29. A method of treating a disorder associated with UDP-3-O—(R-3-hydroxymyristoyl)-N-acetylglucosamine deacetylase (LpxC), said method comprising administering to a patient in need of such treatment a pharmaceutical composition of claim 27.

30. The method of claim 29, wherein said disorder is a microbial infection.

31-38. (canceled)
